(19) United States
(12) Patent Application Publication
(10) Pub. No.: US 2011/0313226 A1
(43) Pub. Date: Dec. 22, 2011

(54) Zeolitic Materials of the LEV-Type Structure And Methods For Their Production

(75) Inventors: Bilge Yilmaz, New York, NY (US); Ulrich Müller, Neustadt (DE); Bibiana Andrea Betancur Moreno, Mannheim (DE); Hermann Gies, Sprockhovel (DE); Feng-Shou Xiao, Changchun (CN); Takashi Tatsumi, Kawasaki (JP); Xinhe Bao, Dalian (CN); Weiping Zhang, Dalian City (CN); Dirk de Vos, Holsbeek (BE); Meike Pfaff, Neunkirchen (DE); Bin Xie, Jilin (CN); Haiyan Zhang, Jilin (CN)

(73) Assignees: Tokyo Institute of Technology, Tokyo (JP); BASF SE, Ludwigshafen (DE)

(21) Appl. No.: 13/163,430

(22) Filed: Jun. 17, 2011

Related U.S. Application Data
(63) Continuation of application No. PCT/CN2010/074073, filed on Jun. 18, 2010.
Application No. PCT/CN2010/074073 is a continuation of application No. PCT/CN2010/078064, filed on Oct. 25, 2010.

(51) Int. Cl. C07C 1/20; C07C 2/00; B01D 53/94; C01B 39/00; B01D 53/56

(52) U.S. Cl. 585/639; 423/700; 423/709; 423/702; 423/713; 423/718; 423/239.2; 423/212; 585/500

(57) ABSTRACT

Described is a process for the production of a zeolitic material having an LEV-type framework structure comprising YO₂ and optionally comprising X₂O₃, wherein said process comprises: (1) preparing a mixture comprising one or more sources for YO₂, one or more solvents, and optionally comprising seed crystals; and (2) crystallizing the mixture obtained in step (1);

wherein Y is a tetravalent element, and X is a trivalent element,
wherein the zeolitic material optionally comprises one or more alkali metals M,
wherein the molar ratio of the total amount of the one or more solvents to the total amount of the one or more sources for YO₂ based on YO₂ is 9.5 or less, and
wherein for crystallization temperatures of 175° C. or higher in step (2), the duration of crystallization at those temperatures is less than 14 d, as well as to a zeolitic material, preferably being obtainable or obtained according to the inventive process, said zeolitic material having an LEV-type framework structure comprising YO₂ and X₂O₃,
wherein the zeolitic material optionally comprises one or more alkali metals M, and wherein the zeolitic material displays an Y:X atomic ratio of from 1 to 9.4.

### CROSS-REFERENCE TO RELATED APPLICATIONS

This application claims benefit under 35 USC 120 and 365(c) of PCT application PCT/CN/2010/074073, filed Jun. 18, 2010 and PCT Application PCT/CN/2010/078064, filed Oct. 25, 2010. The foregoing applications are hereby incorporated herein by reference in their entirety.

### TECHNICAL FIELD

The present invention relates to a zeolitic material having an LEV-type framework structure and to a method for the production of a zeolitic material having an LEV-type framework structure. Furthermore, the present invention relates to the use of such zeolitic materials having an LEV-type framework structure.

### BACKGROUND

The LEV-type framework is characterized by heptadecahedral cavities to which the LEV-type zeolites owe their large micropore volume, although this structure only has small eight-membered ring (8MR) pore openings. The framework density of Levyne is comparable to those of Chabazite (CHA) and Erionite (ERI) having closely related framework structures. Thus, although recent research efforts have focused on large or ultra-large pore zeolites having twelve MR or larger pore openings, small pore zeolites are still of importance because they exhibit zeolite-specific definite shape selectivity with respect to reactant molecules in catalyst applications. In particular, such small pore zeolites having large micropore volumes are attractive due to their large adsorption capacities.

Synthetic Levyne-type zeolites are typically prepared using exotic organotemplates as structure directing agents, such as quinuclidine-based templates, such that their synthesis typically involves high costs. A lower cost alternative is to use diethyldimethylammonium hydroxide as a structure directing agent wherein the diethyldimethylammonium cations act as the organotemplate. Thus, U.S. Pat. No. 7,264,789 B1 discloses a method for preparing LEV-type zeolites which alternatively uses choline and diethyldimethylammonium as organotemplate. A method for the preparation of the LEV-type zeolite RUB-50 using the diethyldimethylammonium cation as oraganotemplate is disclosed in Yamamoto et al. Micropor. Mesopor. Mater. 2010, Vol. 128, pp. 150-157.

Nevertheless, although some progress has been achieved regarding the costs of the organotemplate used in the synthesis of LEV-type zeolites, a major drawback remains with respect to the duration of the synthetic procedure. Thus, Yamamoto et al. reports on a synthetic procedure for the preparation of the LEV-type zeolite RUB-50 which involves hydrothermal crystallization lasting 14 days. U.S. Pat. No. 7,264,789 B1, on the other hand, reports the crystallization of ZSM-45 and NU-3 in a period as short as 4 days, yet at the price of involving an extremely elaborate preparation of specialized seed crystals, which clearly outweighs the benefits of a shorter crystallization period.

Furthermore, the production methods of LEV-type zeolites known from the prior art are limited with respect to the types and properties of the zeolitic materials which may be obtained, in particular with respect to the range of Si:Al molar ratios which are accessible LEV-type aluminosilicates. By way of example, it is noted that the LEV-type zeolite RUB-50 reported in the aforementioned scientific publication from Yamamoto et al. displays an Si:Al molar ratio of 9.5. There is, however, an ongoing need for LEV-type zeolitic materials displaying a higher portion of trivalent elements such as aluminum contained in the framework structure, in particular since these elements often chemically active sites in the zeolitic material, and in particular catalytically active sites which in particular are provided by. Accordingly, regarding for example aluminosilicates belonging to the LEV-type zeolitic materials, it is highly desirable to produce LEV-type framework structures displaying lower Si:Al ratios, and therefore containing a higher portion of chemically, and potentially catalytically active sites at the aluminum positions.

### SUMMARY

The present invention includes the following embodiments, wherein these include the specific combinations of embodiments as indicated by the respective interdependencies defined therein:
1. A process for the production of a zeolitic material having an LEV-type framework structure comprising YO₂ and optionally comprising X₂O₃, wherein said process comprises:
   (1) preparing a mixture comprising one or more sources for YO₂, one or more solvents, and optionally comprising seed crystals; and
   (2) crystallizing the mixture obtained in step (1);
   wherein Y is a tetravalent element, and X is a trivalent element,
   wherein the zeolitic material optionally comprises one or more alkali metals M,
   preferably sodium and/or potassium, and more preferably sodium,
   wherein the molar ratio of the total amount of the one or more solvents to the total amount of the one or more sources for YO₂ based on YO₂ is 9.5 or less, and preferably ranges from 0.5 to 9, more preferably from 1 to 8.5, more preferably from 2 to 8, more preferably from 3 to 7, more preferably from 4 to 6, and even more preferably from 4.5 to 5.5, and
   wherein for crystallization temperatures of 175° C. or higher in step (2), preferably of 170° C. or higher, more preferably of 165° C. or higher, more preferably of 160° C. or higher, and even more preferably of 155° C. or higher, the duration of crystallization at those temperatures is less than 14 d, preferably 13.5 d or less, more preferably 13 d or less, more preferably 10 d or less, more preferably 7 d or less, more preferably 5 d or less, and even more preferably 3.5 d or less.
2. The process of embodiment 1, wherein the one or more solvents comprise one or more polar solvents, wherein the one or more polar solvents are preferably selected from the group consisting of alkanols, water, and mixtures of two or more thereof, more preferably from the group consisting of methanol, ethanol, n-propanol, iso-propanol, water, and mixtures of two or more thereof, more preferably from the group consisting of methanol, ethanol, water, and mixtures of two or more thereof, wherein even more preferably the one or more polar solvents comprise water, preferably distilled water.
3. The process of embodiment 1 or 2, wherein the mixture in step (1) further comprises one or more organotemplates, the one or more organotemplates preferably comprising one or more compounds selected from the group consisting of tetraalkylammonium compounds, 1-methyl-1-azonia-4-azabicyclo[2.2.2]octane, N-methylquinuclidinium compounds, choline compounds, and mixtures of two or more thereof, preferably one or more tetraalkylammonium compounds selected from the group consisting of diethyldimethylammonium compounds, triethylmethylammonium compounds, and mixtures of two or more thereof, wherein more preferably the one or more organotemplates comprises one or more diethyldimethylammonium compounds, more preferably one or more diethyldimethylammonium salts, wherein the one or more diethyldimethylammonium salts are preferably hydroxide and/or halide, wherein more preferably the salts are selected from the group consisting of hydroxide, chloride, bromide, and mixtures of two or more thereof, wherein even more preferably the one or more organotemplates comprises diethyldimethylammonium hydroxide and/or chloride, preferably diethyldimethylammonium hydroxide.
4. The process of embodiment 3, wherein the molar ratio of the total amount of the one or more organotemplates to YO₂ of the mixture obtained in step (1) ranges from 0.01 to 2, preferably from 0.05 to 1, more preferably from 0.1 to 0.8, more preferably from 0.3 to 0.7, more preferably from 0.4 to 0.6, and even more preferably from 0.45 to 0.55.
5. The process of any of embodiments 1 to 4, wherein Y is selected from the group consisting of Si, Sn, Ti, Zr, Ge, and mixtures of two or more thereof, Y preferably being Si.
6. The process of any of embodiments 1 to 5, wherein X is selected from the group consisting of Al, B, In, Ga, and mixtures of two or more thereof, X preferably being Al and/or B, and more preferably being Al.
7. The process of any of embodiments 1 to 6, wherein the one or more sources for YO₂ comprises silica, preferably fumed silica.
8. The process of any of embodiments 1 to 7, wherein the mixture in step (1) further comprises one or more sources for X₂O₃.

9. The process of embodiment 8, wherein the one or more sources for X₂O₃ comprises one or more aluminum compounds, wherein the aluminum compounds are preferably selected from the group consisting of aluminum hydroxide, aluminate salts, and mixtures of two or more thereof, wherein the aluminate salts are preferably alkali metal aluminates, more preferably sodium and/or potassium aluminate, and even more preferably sodium aluminate.
10. The process of embodiment 8 or 9, wherein the YO₂:X₂O₃ molar ratio of the mixture obtained in step (1) ranges from 2 to 200, preferably from 5 to 150, more preferably from 10 to 100, more preferably from 15 to 80, and even more preferably from 20 to 60.
11. The process of any of embodiments 1 to 10, wherein the mixture according to step (1) further comprises one or more sources for OH⁻, wherein said one or more sources for OH⁻ preferably comprises a metal hydroxide, more preferably a hydroxide of an alkali metal M, more preferably sodium and/or potassium hydroxide, and even more preferably sodium hydroxide.
12. The process of embodiment 11, wherein the OH⁻:YO₂ molar ratio of the mixture obtained in step (1) ranges from 0.01 to 5, preferably from 0.05 to 2, more preferably from 0.1 to 1.5, more preferably from 0.2 to 1.1, more preferably from 0.4 to 0.7, and even more preferably from 0.5 to 0.6.
13. The process of any of embodiments 1 to 12, wherein the mixture according to step (1) further comprises one or more sources of one or more elements suitable for isomorphous substitution of at least a portion of the Y atoms and/or of the X atoms in the LEV-type framework structure, wherein the one or more elements are preferably selected from the group consisting of B, Fe, Ti, Sn, Ga, Ge, Zr, V, Nb, Cu, Zn, Li, Be, and mixtures of two or more thereof, more preferably wherein the one or more elements are selected from the group consisting of B, Fe, Ti, Sn, Zr, Cu, and mixtures of two or more thereof, wherein even more preferably the one or more element is Ti and/or B, preferably Ti.
14. The process of embodiment 13, wherein the molar ratio of YO₂ to the total amount of the one or more elements suitable for isomorphous substitution of at least a portion of the Y atoms and/or of the X atoms in the LEV-type framework structure ranges from 3 to 300, preferably from 10 to 200, more preferably from 30 to 150, more preferably from 40 to 100, and even more preferably from 50 to 90.
15. The process of any of embodiments 1 to 14, wherein the M:YO₂ molar ratio of the mixture obtained in step (1) ranges from 0.005 to 1, preferably from 0.01 to 0.5, more preferably from 0.02 to 0.2, more preferably from 0.04 to 0.1, and even more preferably from 0.045 to 0.055.
16. The process of any of embodiments 1 to 15, wherein the YO₂:X₂O₃:M molar ratios of the mixture obtained in step (1) range from 1: (0.005-1): (0.005-1), preferably from 1: (0.01-0.5): (0.01-0.5), more preferably from 1: (0.012-0.2): (0.01-0.5), more preferably from 1: (0.015-0.1): (0.02-0.2), from 1: (0.018-0.07): (0.04-0.1), and even more preferably from 1: (0.02-0.05): (0.045-0.055).
17. The process of any of embodiments 1 to 16, wherein the crystallization in step (2) involves heating of the mixture, preferably at a temperature ranging from 50 to 250° C., more preferably from 80 to 200° C., more preferably from 100 to 180° C., more preferably from 120 to 170° C., more preferably from 140 to 160° C., and even more preferably from 145 to 155° C.
18. The process of embodiment 17, wherein the crystallization in step (2) is conducted under solvothermal conditions, preferably under hydrothermal conditions.
19. The process of any of embodiments 1 to 18, wherein the crystallization in step (2) involves heating of the mixture for at least 0.1 d, and preferably for a period ranging from 0.5 to 50 d, preferably from 1 to 30 d, more preferably from 1.5 to 13 d, more preferably from 2 to 10 d, more preferably from 2 to 7 d, more preferably from 2.5 to 5 d, and even more preferably from 2.5 to 3.5 d.
20. The process of any of embodiments 1 to 19, wherein the crystallization in step (2) involves agitating the mixture, preferably by stirring and/or rotation, more preferably by stirring of the mixture obtained in step (1).
21. The process of any of embodiments 1 to 20, further comprising one or more of the following steps of
   (3) isolating the zeolitic material having an LEV-type framework structure, preferably by filtration, ultrafiltration, diafiltration, centrifugation and/or decantation methods,
   and/or
   (4) washing the zeolitic material having an LEV-type framework structure, and/or
   (5) drying and/or calcining the zeolitic material having an LEV-type framework structure,
   and/or
   (6) subjecting the zeolitic material having an LEV-type framework structure to an ion-exchange procedure,
   wherein the steps (3) and/or (4) and/or (5) and/or (6) can be conducted in any order, and wherein one or more of said steps is preferably repeated one or more times.
22. The process of embodiment 21, wherein the calcination in step (5) is conducted at a temperature in the range of from 300 to 900° C., preferably from 400 to 800° C., more preferably from 500 to 700° C., and even more preferably from 550 to 650° C.
23. The process of embodiment 21 or 22, wherein after step (2) and prior to step (3) the pH of the crystallization product is adjusted to a pH in the range of from 5 to 12, preferably from 6 to 11, more preferably from 7 to 10, more preferably from 8 to 9.5, and even more preferably to a pH in the range of from 8.3 to 9.3.
24. The process of any of embodiments 21 to 23, wherein in the one or more step (6) at least one ionic non-framework element contained in the zeolitic material having an LEV-type framework is ion-exchanged, preferably against at least one cation and/or cationic element, wherein at least one cation and/or cationic element is preferably selected from the group consisting of H⁺, NH₄⁺, Sr, Zr, Cr, Fe, Co, Ni, Cu, Zn, Ru, Rh, Pd, Ag, Os, Ir, Pt, Au, and mixtures of two or more thereof, more preferably from the group consisting of H⁺, NH₄⁺, Sr, Cr, Fe, Co, Ni, Cu, and mixtures of two or more thereof, and even more preferably from the group consisting of H⁺, NH₄⁺, Fe, Cu, and mixtures of two or more thereof,
   wherein said at least one ionic non-framework element is preferably one or more alkali metals M, preferably sodium and/or potassium, and more preferably sodium.
25. The process of any of embodiments 1 to 24, wherein the zeolitic material having an LEV-type framework structure formed in step (2) comprises one or more zeolites selected from the group consisting of Levyne, LZ-132, NU-3, RUB-1, ZK-20, ZSM-45, RUB-50, and mixtures of two or more thereof, wherein preferably the zeolitic material comprises RUB-50.
26. The process of any of embodiments 1 to 25, wherein the seed crystals at least partially comprise zeolitic material having an LEV-type framework structure, wherein the zeolitic material having an LEV-type framework structure is preferably the zeolitic material obtained or obtainable according to any one of embodiments 1 to 25.
27. The process of embodiment 26, wherein the zeolitic material having an LEV-type framework structure contained in the seed crystals comprises one or more zeolites selected from the group consisting of Levyne, LZ-132, NU-3, RUB-1, ZK-20, ZSM-45, RUB-50, and mixtures of two or more thereof, wherein the seed crystals preferably comprise RUB-50.
28. The process of any of embodiments 1 to 25, wherein the seed crystals at least partially comprise zeolitic material not having an LEV-type framework structure, wherein preferably the seed crystals do not comprise zeolitic material having an LEV-type framework structure, wherein the zeolitic material not having an LEV-type framework material preferably comprises zeolitic material having a CHA-type framework structure.
29. The process of embodiment 28, wherein the zeolitic material having a CHA-type framework structure contained in the seed crystals comprises chabazite and/or SSZ-13, preferably SSZ-13.
30. The process of any of embodiments 1 to 29, wherein the amount of seed crystals in the mixture according to step (1) ranges from 0.01 to 30 wt.-% based on 100 wt.-% of YO₂ in the at least one source for YO₂, preferably from 0.1 to 20 wt.-%, more preferably from 0.5 to 10 wt.-%, more preferably from 2 to 8 wt.-%, and even more preferably from 3 to 5 wt.-% based on 100 wt.-% of YO₂.
31. The process of any of embodiments 1 to 29, wherein the mixture according to step (1) contains 5 wt.-% or less of seed crystals based on 100 wt.-% of YO₂, preferably 1 wt.-% or less, more preferably 0.5 wt.-% or less, more preferably 0.1 wt.-% or less, more preferably 0.01 wt.-% or less, and wherein even more preferably, the mixture according to step (1) contains no seed crystals.
32. A zeolitic material having an LEV-type framework structure obtainable and/or obtained according to the process of any one of embodiments 1 to 31.
33. A zeolitic material having an LEV-type framework structure, preferably obtainable and/or obtained according to the process of any one of embodiments 1 to 31, said zeolitic material comprising YO₂ and X₂O₃,
   wherein Y is a tetravalent element, and X is a trivalent element,
   wherein the zeolitic material optionally comprises one or more alkali metals M, preferably sodium and/or potassium, and more preferably sodium, and wherein the zeolitic material displays an Y:X atomic ratio of from 1 to 9.4, preferably from 2 to 9.3, more preferably from 3 to 9.1, more preferably from 5 to 9.0, more preferably from 7.5 to 8.9, more preferably from 8 to 8.8, and even more preferably from 8.5 to 8.7.
34. The zeolitic material of embodiment 32 or 33, wherein Y is selected from the group consisting of Si, Sn, Ti, Zr, Ge, and mixtures of two or more thereof, Y preferably being Si.
35. The zeolitic material of any of embodiments 32 to 34, wherein X is selected from the group consisting of Al, B, In, Ga, and mixtures of two or more thereof, X preferably being Al and/or B, and more preferably being Al.
36. The zeolitic material of any of embodiments 32 to 35, said material having an X-ray diffraction pattern comprising at least the following reflections:

| Intensity (%) | Diffraction angle 2θ/° [Cu K(alpha 1)] |
|---|---|
| [26-40] | [10.94-11.20] |
| [23-27] | [13.36-13.62] |
| [50-66] | [17.33-17.62] |
| [42-57] | [21.05-21.37] |
| 100 | [22.08-22.21] |
| [35-56] | [28.59-28.92] |
| [48-62] | [32.37-32.60] |
| [12-19] | [51.60-52.14] |
| [11-13] | [55.68-56.32] |

wherein 100% relates to the intensity of the maximum peak in the X-ray powder diffraction pattern.
37. The zeolitic material of any of embodiments 32 to 36, wherein at least a portion of the Y atoms and/or of the X atoms in the LEV-type framework structure is isomorphously substituted by one or more elements, wherein the one or more elements are preferably selected from the group consisting of B, Fe, Ti, Sn, Ga, Ge, Zr, V, Nb, Cu, Zn, Li, Be, and mixtures of two or more thereof, more preferably wherein the one or more elements are selected from the group consisting of B, Fe, Ti, Sn, Zr, Cu, and mixtures of two or more thereof, wherein even more preferably the one or more element is Ti and/or B, preferably Ti.
38. The zeolitic material of embodiment 37, wherein the molar ratio of YO₂ to the total amount of the one or more elements by which the LEV-type framework structure is isomorphously substituted ranges from 5 to 100, preferably from 10 to 80, more preferably from 20 to 70, and even more preferably from 25 to 65.
39. The zeolitic material of any of embodiments 32 to 38, wherein at least a portion of the alkali metal atoms M is substituted by one or more cation and/or cationic element, wherein the one or more cation and/or cationic element is preferably selected from the group consisting of H⁺, NH₄⁺, Sr, Zr, Cr, Fe, Co, Ni, Cu, Zn, Ru, Rh, Pd, Ag, Os, Ir, Pt, Au, and mixtures of two or more thereof, more preferably from the group consisting of H⁺, NH₄⁺, Sr, Cr, Fe, Co, Ni, Cu, and mixtures of two or more thereof, and even more preferably from the group consisting of H⁺, NH₄⁺, Fe, Cu, and mixtures of two or more thereof.
40. The zeolitic material of any of embodiments 32 to 39, wherein said material comprises one or more zeolites selected from the group consisting of Levyne, LZ-132, NU-3, RUB-1, ZK-20, ZSM-45, RUB-50, and mixtures of two or more thereof, wherein preferably the zeolitic material comprises RUB-50.
41. Use of a zeolitic material of any of embodiments 32 to 40 as a molecular sieve, catalyst, catalyst support, and/or as an adsorbent, wherein the zeolitic material is preferably used as a molecular trap for chemical compounds, as a catalyst and/or as a catalyst support.

### BRIEF DESCRIPTION OF THE DRAWINGS

FIGS. 1, 2, 3B, and 4A show the X-ray diffraction pattern of the crystalline material obtained according to Examples 1, 2, 3, and 4, respectively.

FIG. 3A shows the X-ray diffraction pattern of the RUB-50 seed crystals used in Example 3.

FIGS. 3C and 4B show the scanning electron microscope (SEM) images obtained from samples of the crystalline products obtained according to Examples 3 and 4, respectively.

### DETAILED DESCRIPTION

According to one or more embodiments of the present invention an improved process for the production of a zeolitic material having an LEV-type framework structure is provided, in particular with respect to the production efficiency and/or with respect to the type of products which may be accessed by the production process. In one or more embodiments, an improved zeolitic material is provided, in particular with respect to its potential applications including, for example, its use as a molecular sieve and/or as a catalyst or also as a catalyst support.

Thus, the an embodiment of the present invention relates to a process for the production of a zeolitic material having an LEV-type framework structure comprising YO₂ and optionally comprising X₂O₃, wherein said process comprises:
(1) preparing a mixture comprising one or more sources for YO₂, one or more solvents, and optionally comprising seed crystals; and
(2) crystallizing the mixture obtained in step (1);
   wherein Y is a tetravalent element, and X is a trivalent element,
   wherein the zeolitic material optionally comprises one or more alkali metals M, preferably sodium and/or potassium, and more preferably sodium,
   wherein the molar ratio of the total amount of the one or more solvents to the total amount of the one or more sources for YO₂ based on YO₂ is 9.5 or less, and preferably ranges from 0.5 to 9, more preferably from 1 to 8.5, more preferably from 2 to 8, more preferably from 3 to 7, more preferably from 4 to 6, and even more preferably from 4.5 to 5.5, and wherein for crystallization temperatures of 175° C. or higher in step (2), preferably of 170° C. or higher, more preferably of 165° C. or higher, more preferably of 160° C. or higher, and even more preferably of 155° C. or higher, the duration of crystallization at those temperatures is less than 14 d, preferably 13.5 d or less, more preferably 13 d or less, more preferably 10 d or less, more preferably 7 d or less, more preferably 5 d or less, and even more preferably 3.5 d or less.

In particular, it has surprisingly been found that by using molar ratios comprised in a specific range of the total amount of the one or more solvents to the total amount of the one or more sources for YO₂ based on YO₂, it is possible to considerably shorten the duration of crystallization independently of the additional use of seed crystals in the reaction mixture. Furthermore and quite unexpectedly, it has been found that the use of molar ratios comprised in said specific range also allows for the synthesis of a zeolitic material displaying exceptionally low molar ratios of the trivalent element X to the tetravalent element Y present in the final product, and in particular to very low Si:Al molar ratios in aluminosilicate zeolites having an LEV-type framework structure.

It is herewith noted that within the meaning of the present invention, and in particular with respect to specific embodiments thereof, the term “comprising” is preferably used as meaning “consisting of”. Furthermore, within the meaning of an embodiment of the present invention, references to the molar content of YO₂ and X₂O₃ and to molar ratios thereof as contained in mixtures provided in the inventive process or in the zeolites described herein generally refer to the respective molar contents of Y and X on the basis of YO₂ and X₂O₃ as commonly employed in zeolite chemistry. In particular, references to YO₂ and X₂O₃ does not imply that said specific compounds must actually be contained in mixtures provided in the inventive process or in the zeolites, but rather refer to the molar amounts of Y and X present therein in terms of YO₂ and X₂O₃, respectively.

Furthermore, according to embodiments and preferred embodiments of the present invention, YO₂ and X₂O₃ are comprised in the LEV-type framework structure as structure building elements, as opposed to non-framework elements which can be present in the pores and cavities formed by the framework structure and typical for zeolitic materials in general. According to the inventive process, a zeolitic material having an LEV-type framework structure is crystallized in step (2). Said material comprises YO₂, wherein Y stands for any conceivable tetravalent element, Y standing for either one or several tetravalent elements. Preferred tetravalent elements according to an embodiment of the present invention include Si, Sn, Ti, Zr, and Ge, and combinations thereof. More preferably, Y stands for Si, Ti, or Zr, or any combination of said trivalent elements, even more preferably for Si and/or Sn. According to the present invention, it is particularly preferred that Y stands for Si.

Thus, in principle, any conceivable zeolitic material having an LEV-type framework structure may be formed in step (2) of the inventive process, provided that it comprises YO₂ as a framework element, wherein Y stands for any conceivable tetravalent element. According to preferred embodiments of the inventive process wherein YO₂ comprises SiO₂, it is further preferred that the zeolitic material having an LEV-type framework structure formed in step (2) comprises one or more zeolites selected from the group consisting of Levyne, LZ-132, NU-3, RUB-1, ZK-20, ZSM-45, RUB-50, and mixtures of two or more thereof, wherein even more preferably the zeolitic material comprises RUB-50.

Furthermore, according to the process of an embodiment of the present invention one or more sources for YO₂ can be provided in step (1) in any conceivable form, provided that a zeolitic material having an LEV-type framework structure comprising YO₂ can be crystallized in step (2). Preferably, YO₂ is provided as such and/or as a compound which comprises YO₂ as a chemical moiety and/or as a compound which (partly or entirely) is chemically transformed to YO₂ during the inventive process. In preferred embodiments of the present invention, wherein Y stands for Si or for a combination of Si with one or more further tetravalent elements. The source for SiO₂ preferably provided in step (1) can be any conceivable source. Preferably, YO₂ is provided as such and/or as a compound which comprises YO₂ as a chemical moiety and/or as a compound which (partly or entirely) is chemically transformed to YO₂ during the inventive process. There can therefore be used, for example, all types of silica and silicates, preferably fumed silica, silica hydrosols, reactive amorphous solid silicas, silica gel, silicic acid, water glass, sodium metasilicate hydrate, sesquisilicate or disilicate, colloidal silica, pyrogenic silica, silicic acid esters, or tetraalkoxysilanes, or mixtures of at least two of these compounds.

In preferred embodiments of the inventive process, wherein the mixture according to step (1) comprises at least one source for SiO₂, said source preferably comprises at least one compound selected from the group consisting of silica and silicates. Among the silicates which may be employed, alkali metal silicates are preferred, more preferably water glass, more preferably sodium and/or potassium silicate, and even more preferably sodium silicate. Among the silica which may be employed, fumed silica is particularly preferred. According to particularly preferred embodiments the at least one source for SiO₂ comprises silica, preferably fumed silica.

Further preferred are embodiments wherein the zeolitic material having an LEV-type framework structure further comprises X₂O₃, wherein X stands for any conceivable trivalent element, X standing for either one or several trivalent elements. Preferred tetravalent elements according to an embodiment of the present invention include Al, B, In, and Ga, and combinations thereof. More preferably, Y stands for Al, B, or In, or any combination of said trivalent elements, even more preferably for Al and/or B. According to an embodiment of the present invention, it is particularly preferred that X stands for Al.

According to preferred embodiments of the present invention, wherein the zeolitic material having an LEV-type framework structure comprises X₂O₃, one or more sources for X₂O₃ are provided in step (1). In general, X₂O₃ can be provided in any conceivable form, provided that a zeolitic material having an LEV-type framework structure comprising X₂O₃ can be crystallized in step (2). Preferably, X₂O₃ is provided as such and/or as a compound which comprises X₂O₃ as a chemical moiety and/or as a compound which (partly or entirely) is chemically transformed to X₂O₃ during the inventive process.

In more preferred embodiments of the present invention, wherein X stands for Al or for a combination of Al with one or more further trivalent elements, the one or more sources for Al₂O₃ preferably provided in step (1) can be selected from any conceivable source. There can be used for example any type of alumina; aluminate salts such as, for example, alkali metal aluminates and preferably sodium and/or potassium aluminate, more preferably sodium aluminate; aluminum alcoholates, such as, for example, aluminum triisopropylate; at least partially hydrated alumina such as, for example, AlO(OH), and aluminum hydroxide; as well as mixtures of two or more of any of the aforementioned examples for sources of Al₂O₃. Preferably, the one or more sources for Al₂O₃ comprises one or more aluminum alcoholates and/or one or more at least partially hydrated aluminas, more preferably one or more at least partially hydrated aluminas, wherein even more preferably the one or more sources for Al₂O₃ comprises aluminum hydroxide, and in particular Al(OH)₃.

According to embodiments of the present invention which are further preferred, wherein X stands for Al or for a combination of Al with one or more further trivalent elements, the one or more sources for Al₂O₃ preferably provided in step (1) is selected from the group consisting of aluminum hydroxide, aluminate salts, and mixtures of two or more thereof, wherein the aluminate salts are preferably alkali metal aluminates, more preferably sodium and/or potassium aluminate, and even more preferably sodium aluminate. According to particularly preferred embodiments thereof, the one or more sources for Al₂O₃ preferably provided in step (1) comprise one or more aluminate salts, preferably alkali metal aluminates, more preferably sodium and/or potassium aluminate, and even more preferably sodium aluminate.

Therefore, it is further preferred according to an embodiment of the present invention that the one or more sources for X₂O₃ comprises one or more aluminum compounds, wherein the aluminum compounds are preferably selected from the group consisting of aluminum hydroxide, aluminate salts, and mixtures of two or more thereof, wherein the aluminate salts are preferably alkali metal aluminates, more preferably sodium and/or potassium aluminate, and even more preferably sodium aluminate.

In embodiments wherein X stands for B or for a combination of B with one or more trivalent elements and preferably Al, the one or more sources for B₂O₃ provided in step (1) may also be selected from practically any conceivable source. Examples thereof include free boric acid, borates, and boric esters such as, for example, triethyl borate or trimethyl borate, in addition to combinations of two or more thereof.

According to particularly preferred embodiments of the inventive process, the mixture according to step (1) comprises at least one silica as a source for YO₂ and at least one at least partially hydrated alumina as a source for X₂O₃, and more preferably at least one fumed silica and/or at least one of AlO(OH) and/or Al(OH)₃, wherein even more preferably the mixture according to (1) comprises at least one fumed silica and Al(OH)₃.

In preferred embodiments of the inventive process wherein the mixture obtained in step (1) comprises at least one source for X₂O₃, the YO₂:X₂O₃ molar ratio of the mixture can have any conceivable value, provided that a zeolitic material having an LEV-type framework structure comprising both YO₂ and X₂O₃ is crystallized in step (2). Generally, the molar ratio may range anywhere from 2 to 200, and preferably from 5 to 150, more preferably from 10 to 100, more preferably from 15 to 80, and even more preferably from 20 to 60.

According to certain embodiments of the present invention, the zeolitic material obtained and/or obtainable according to the inventive process and/or the inventive material as such comprises one or more alkali metals M, preferably sodium and/or potassium, and more preferably sodium. The alkali metal can be added at any conceivable stage of the inventive process, wherein preferably it is also added in step (1). More preferably, the entire quantity of the alkali metal comprised in the zeolitic material having an LEV-type framework structure is added in step (1) of the inventive process. In general, the one or more alkali metals may be added in any conceivable form, wherein it is preferably comprised in the at least one source for X₂O₃ and/or YO₂ and/or wherein it is added as a salt in addition to the at least one source for X₂O₃ and/or YO₂, wherein more preferably the one or more alkali metals are comprised in the at least one source for X₂O₃ and/or they are added as a salt instead of or in addition thereto, wherein even more preferably the one or more alkali metals are added as a metal salt in addition to the one or more sources for X₂O₃ and YO₂. In embodiment, wherein the one or more alkali metals are added in addition to the one or more sources for X₂O₃ and YO₂, it is generally added as a metal salt, wherein any suitable alkali metal salt may be employed, and wherein preferably alkali metal halides and/or alkali metal hydroxides may be used, and wherein preferably alkali metal hydroxides are employed, more preferably sodium and/or potassium hydroxide, and even more preferably sodium hydroxide. In instances wherein the one or more alkali metals are comprised in the one or more source for X₂O₃, and wherein said one or more sources for X₂O₃ comprise Al, the one or more alkali metals are preferably comprised in one or more aluminate salts. In particularly preferred embodiments of the inventive process, the alkali metal is added as a metal salt in addition to the at least one source for YO₂ and/or X₂O₃ provided in step (1), more preferably as alkali metal hydroxide, more preferably as sodium and/or potassium hydroxide, and even more preferably as sodium hydroxide.

In general, the alkali metal M can be contained in the mixture according to step (1) of the inventive process in any conceivable amount, provided that a zeolitic material having an LEV-type framework structure is crystallized in step (2). Preferably, the M:YO₂ molar ratio of the mixture obtained in step (1) ranges from 0.005 to 1, preferably from 0.01 to 0.5, more preferably from 0.02 to 0.2, more preferably from 0.04 to 0.1, and even more preferably from 0.045 to 0.055.

According to preferred embodiments of the inventive process, the mixture according to step (1) comprises at least one source for X₂O₃ and at least one alkali metal M. In general, any conceivable amounts of these components can be contained in the mixture provided that a zeolitic material having an LEV-type framework structure is crystallized in step (2). Preferably, the YO₂:X₂O₃:M molar ratios of the mixture obtained in step (1) range from 1: (0.005-1): (0.005-1), preferably from 1: (0.01-0.5): (0.01-0.5), more preferably from 1: (0.012-0.2): (0.01-0.5), more preferably from 1: (0.015-0.1): (0.02-0.2), from 1: (0.018-0.07): (0.04-0.1), and even more preferably from 1: (0.02-0.05): (0.045-0.055).

According to the process of an embodiment of the present invention, the mixture provided in step (1) can contain one or more sources for hydroxide anions OH⁻. In general any conceivable source for OH⁻ can be used, wherein the at least one source preferably comprises a metal hydroxide, more preferably a hydroxide of an alkali metal M, more preferably sodium and/or potassium hydroxide, and even more preferably sodium hydroxide. Within a preferred meaning of an embodiment of the present invention, the term “source for OH⁻” refers to chemical compounds and compositions which may be solvated in the one or more solvents provided in step (1) of the inventive process thus leading to the formation of at least partially solvated hydroxide ions and/or wherein hydroxide ions may be generated and dissociated from said one or more sources for OH⁻ in the course of the inventive process, including in the course of the crystallization procedure in step (2).

In general the OH⁻:YO₂ molar ratio of the mixture obtained in step (1) of the inventive process can have any conceivable value, provided that a zeolitic material having an LEV-type framework structure is crystallized in step (2). Preferably, the OH:YO₂ molar ratio ranges from 0.01 to 5, preferably from 0.05 to 2, more preferably from 0.1 to 1.5, more preferably from 0.2 to 1.1, more preferably from 0.4 to 0.7, and even more preferably from 0.5 to 0.6.

According to preferred embodiments of the inventive process, seed crystals are provided in step (1). In general, any conceivable seed crystals or mixtures thereof may be provided therein, provided that a zeolitic material having an LEV-type framework structure comprising YO₂ and optionally comprising X₂O₃ may be obtained in step (2).

According to certain preferred embodiments of the inventive process, the seed crystals provided in step (1) at least partially comprise zeolitic material having an LEV-type framework structure. In general, said seed crystals can comprise any zeolitic material having an LEV-type framework structure, provided that a zeolitic material having an LEV-type framework structure is crystallized in step (2). Preferably, the zeolitic material having an LEV-type framework structure comprised in the seed crystals is a zeolitic material obtained according to the inventive process. More preferably, the zeolitic material having an LEV-type framework structure comprised in the seed crystals is the same as the zeolitic material having an LEV-type framework structure which is then crystallized in step (2). Particularly preferred according to an embodiment of the present invention are seed crystals comprising one or more zeolites selected from the group consisting of Levyne, LZ-132, NU-3, RUB-1, ZK-20, ZSM-45, RUB-50, and mixtures of two or more thereof, wherein even more preferably the seed crystals preferably comprise RUB-50. According to an even more preferred embodiment the seed crystals comprise one or more zeolites selected from the group consisting of Levyne, LZ-132, NU-3, RUB-1, ZK-20, ZSM-45, RUB-50, and mixtures of two or more thereof, wherein said one or more zeolites have been obtained according to the inventive process, wherein even more preferably RUB-50 obtained according to the inventive process is comprised in the seed crystals.

According to alternatively preferred embodiments of the inventive process, the seed crystals provided in step (1) at least partially comprise zeolitic material not having an LEV-type framework structure, wherein preferably the seed crystals do not comprise zeolitic material having an LEV-type framework structure. In general, said seed crystals can comprise any zeolitic material other than zeolitic materials having an LEV-type framework structure, provided that a zeolitic material having an LEV-type framework structure may be obtained in step (2). Thus, according to a further aspect of the present invention, it has quite surprisingly been found that it is possible to produce a zeolitic material having an LEV-type framework structure using seed crystals not displaying the LEV-type framework structure of the zeolitic material obtained in step (2). This result was not only completely unexpected, but also provides considerable advantages since the general advantages linked to the use of a seeding material and, in particular to the use of seed crystals, may be achieved using heteroseeds which may be far easier to obtain and/or to manufacture, as a result of which the cost-effectiveness of the inventive process may be yet improved. According to a particularly preferred embodiment of the inventive process which employs seed crystals which at least in part do not comprise zeolitic material having an LEV-type framework structure, and which preferably do not comprise zeolitic material having an LEV-type framework structure, the seed crystals employed in said particularly preferred embodiments preferably comprise zeolitic material having a CHA-type framework structure, wherein more preferably the zeolitic material having a CHA-type framework structure contained in the seed crystals comprises chabazite and/or SSZ-13, and even more preferably SSZ-13

According to the inventive process, any suitable amount of seed crystals can be optionally provided in the mixture according to step (1), provided that a zeolitic material having an LEV-type framework structure is crystallized in step (2). According to a preferred embodiment, the amount of seed crystals contained in the mixture according to step (1) ranges from 0.01 to 30 wt.-% based on 100 wt.-% of YO₂ in the at least one source for YO₂, and preferably from 0.1 to 20 wt.-%, more preferably from 0.5 to 10 wt.-%, more preferably from 2 to 8 wt.-%, and even more preferably from 3 to 5 wt.-% based on 100 wt.-% of YO₂. According to an alternatively preferred embodiment of the inventive process, the mixture according to step (1) contains 5 wt.-% or less of seed crystals based on 100 wt.-% of YO₂, preferably 1 wt.-% or less, more preferably 0.5 wt.-% or less, more preferably 0.1 wt.-% or less, more preferably 0.01 wt.-% or less, and wherein even more preferably, the mixture according to step (1) contains no seed crystals.

In step (1) according to an embodiment of the present invention, the mixture can be prepared by any conceivable means, wherein mixing by agitation is preferred, preferably by means of stirring.

According to an embodiment of the present invention, the mixture obtained in step (1) of the inventive process further comprises one or more solvents. In principle any conceivable solvents may be used, provided that a zeolitic material having an LEV-type framework structure can be crystallized in step (2). According to preferred embodiments, the one or more solvents comprise one or more polar solvents. In general, any polar solvent may be used including protic and aprotic solvents as well as combinations thereof, wherein solvents or solvent mixtures are preferably used which comprise one or more protic solvents. In particular it is preferred that the one or more solvents comprise one or more polar solvents selected from the group consisting of alkanols, water, and mixtures of two or more thereof, more preferably from the group consisting of methanol, ethanol, n-propanol, iso-propanol, water, and mixtures of two or more thereof, more preferably from the group consisting of methanol, ethanol, water, and mixtures of two or more thereof, wherein even more preferably the one or more polar solvents comprise water, and preferably distilled water.

Furthermore, the molar ratio of the total amount of the one or more solvents to the total amount of the one or more sources for YO₂ based on YO₂ as provided in step (1) is 9.5 or less, and is preferably comprised in the range of from 0.5 to 9, more preferably of from 1 to 8.5, more preferably of from 2 to 8, more preferably of from 3 to 7, more preferably of from 4 to 6, and wherein even more preferably the molar ratio of the total amount of the one or more solvents to the total amount of the one or more sources for YO₂ based on YO₂ is comprised in the range of from 4.5 to 5.5.

According to the inventive process, the mixture provided in step (1) may contain any further compound or material which may suitably be used for the crystallization of a zeolitic material having an LEV-type framework structure in step (2). In particular, according to preferred embodiments of the present invention, the mixture in step (1) further comprises one or more organotemplates which may suitable serve as structure directing agents in the crystallization process. In general, any structure directing agent and, in particular, any organotemplate may be used in the present invention, provided that a zeolitic material having an LEV-type framework structure may be obtained. According to the inventive process, it is preferred that the one or more organotemplates comprise one or more compounds selected from the group consisting of tetraalkylammonium compounds, 1-methyl-1-azonia-4-azabicyclo[2.2.2]octane, N-methylquinuclidinium compounds, choline compounds, and mixtures of two or more thereof, wherein preferably the one or more tetraalkylammonium compounds are selected from the group consisting of diethyldimethylammonium compounds, triethylmethylammonium compounds, and mixtures of two or more thereof. More preferably, the one or more organotemplates comprise one or more diethyldimethylammonium compounds, more preferably one or more diethyldimethylammonium salts. In principle, any diethyldimethylammonium salt may be used, wherein preferably diethyldimethylammonium hydroxide and/or one or more diethyldimethylammonium halides are used, more preferably the one or more diethyldimethylammonium salts selected from the group consisting of hydroxide, chloride, bromide, and mixtures of two or more thereof, wherein even more preferably the one or more organotemplates comprise diethyldimethylammonium hydroxide and/or chloride, and preferably diethyldimethylammonium hydroxide.

With respect to preferred embodiments of the present invention wherein the mixture in step (1) comprises one or more organotemplates, suitably as structure directing agents, there is no particular restriction as to the total amount of organotemplates which may be used, provided that a zeolitic material having an LEV-type framework structure according to the embodiments and preferred embodiments of the present invention may be obtained. Thus, by way of example, the molar ratio of the total amount of the one or more organotemplates to YO₂ of the mixture obtained in step (1) may range anywhere from 0.01 to 2, wherein preferably the total amount of the one or more organotemplates used preferably ranges from 0.05 to 1, more preferably from 0.1 to 0.8, more preferably from 0.3 to 0.7, more preferably from 0.4 to 0.6, and even more preferably from 0.45 to 0.55.

Furthermore, according preferred embodiments of the inventive process, the mixture according to step (1) comprises one or more sources of one or more elements suitable for isomorphous substitution of at least a portion of the Y atoms and/or of the X atoms in the LEV-type framework structure. In general, any conceivable elements can be used provided that they may effectively be substituted into the LEV-type framework structure via isomorphous substitution. In preferred embodiments, the one or more elements are selected from the group consisting of B, Fe, Ti, Sn, Ga, Ge, Zr, V, Nb, Cu, Zn, Li, Be, and mixtures of two or more thereof, more preferably wherein the one or more elements are selected from the group consisting of B, Fe, Ti, Sn, Zr, Cu, and mixtures of two or more thereof, wherein even more preferably the one or more element is Ti and/or B, preferably Ti. According to further embodiments of the present invention which are particularly preferred, the one or more elements comprise Cu, wherein it is particularly preferred that the one or more element suitable for isomorphous substitution is Cu. According to yet further embodiments of the present invention which are particularly preferred, the one or more elements comprise Fe, wherein it is particularly preferred that the one or more element suitable for isomorphous substitution is Fe.

Therefore, an embodiment of the present invention therefore also provides a one-pot synthetic procedure for the preparation of a zeolitic material having an LEV-type framework structure which is isomorphously substituted, wherein isomorphous substitution is not achieved by conventional processes involving the post-synthetic treatment of an existing framework, wherein framework elements are treated such that they may be replaced with other atoms which are then contained in the resulting framework structure. In particular, according to the inventive process it is not necessary to remove existing framework atoms for producing an isomorphously substituted framework structure.

Consequently, an embodiment of the present invention also relates to a one-pot synthetic procedure for the production of a zeolitic material having an LEV-type framework structure, wherein at least a portion of the Y atoms and/or of the X atoms in the LEV-type framework structure is isomorphously substituted by one or more elements, wherein the one or more elements are preferably selected from the group consisting of B, Fe, Ti, Sn, Ga, Ge, Zr, V, Nb, Cu, Zn, Li, Be, and mixtures of two or more thereof, more preferably wherein the one or more elements are selected from the group consisting of B, Fe, Ti, Sn, Zr, Cu, and mixtures of two or more thereof, wherein even more preferably the one or more element is Ti and/or B, preferably Ti, wherein according to embodiments which are further preferred the one or more elements used for isomorphous substitution is Cu and/or Fe.

In general, according to step (1) of the inventive process, the molar ratio of YO₂ to the element or to the sum of the one or more elements suitable for isomorphous substitution can have any conceivable value, wherein the molar ratio preferably ranges from 3 to 300, preferably from 10 to 200, more preferably from 30 to 150, more preferably from 40 to 100, and even more preferably from 50 to 90.

In general, step (2) according to the inventive process can be conducted in any conceivable manner, provided that a zeolitic material having an LEV-type framework structure is crystallized from the mixture according to step (1). The mixture can be crystallized in any suitable type of vessel or receptacle, wherein a means of agitation is preferably employed, preferably by rotation of the vessel and/or stirring, and more preferably by stirring the mixture.

According to the inventive process, the mixture is preferably heated during at least a portion of the crystallization process in step (2). In general, the mixture can be heated to any conceivable temperature of crystallization, provided that a zeolitic material having an LEV-type framework structure is crystallized from the mixture. Thus, by way of example, the mixture may be heated in step (2) to a temperature comprised in the range of from 50 to 250° C., wherein preferably the mixture is heated in step (2) to a temperature of crystallization ranging from 80 to 200° C., more preferably from 100 to 180° C., more preferably from 120 to 170° C., more preferably from 140 to 160° C., and even more preferably from 145 to 155° C.

In preferred embodiments of the present invention, the mixture according to step (1) is subjected in step (2) to a pressure which is elevated with regard to normal pressure. The term “normal pressure” as used in the context of an embodiment of the present invention relates to a pressure of 101,325 Pa in the ideal case. However, this pressure may vary within boundaries known to the person skilled in the art. By way of example, this pressure can be in the range of from 95,000 to 106,000 or of from 96,000 to 105,000 or of from 97,000 to 104,000 or of from 98,000 to 103,000 or of from 99,000 to 102,000 Pa.

In preferred embodiments of the inventive process wherein a solvent is present in the mixture according to step (1), it is furthermore preferred that heating in step (2) is conducted under solvothermal conditions, meaning that the mixture is crystallized under autogenous pressure of the solvent which is used, for example by conducting heating in an autoclave or other crystallization vessel suited for generating solvothermal conditions. In particularly preferred embodiments wherein the solvent comprises water, preferably distilled water, heating in step (2) is accordingly preferably conducted under hydrothermal conditions.

The apparatus which can be used in the present invention for crystallization is not particularly restricted, provided that the desired parameters for the crystallization process can be realized, in particular with respect to the preferred embodiments requiring particular crystallization conditions. In the preferred embodiments conducted under solvothermal conditions, any type of autoclave or digestion vessel can be used, wherein a Teflon-lined apparatus is preferred.

In general, the duration of the crystallization process in step (2) of the inventive process is not particularly limited. Thus, the crystallization process may be conducted for any conceivable duration, provided that a zeolitic material having an LEV-type framework structure according to embodiments and preferred embodiments of the present invention is provided. Accordingly, in principle, the crystallization may be conducted for at least 0.1 d, wherein the mixture is preferably heated. In further preferred embodiments involving heating of the mixture obtained in step (1), said crystallization process is preferably conducted for a period ranging from 0.5 to 50 d, more preferably from 1 to 30 d, more preferably from 1.5 to 13 d, more preferably from 2 to 10 d, more preferably from 2 to 7 d, more preferably from 2.5 to 5 d, and even more preferably from 2.5 to 3.5 d.

Nevertheless, according to preferred embodiments of the present invention, the inventive process is preferably provided with the proviso that for crystallization temperatures of 175° C. or higher in step (2), preferably of 170° C. or higher, more preferably of 165° C. or higher, more preferably of 160° C. or higher, and even more preferably of 155° C. or higher, the duration of crystallization at those temperatures is less than 14 d, preferably 13.5 d or less, more preferably 13 d or less, more preferably 10 d or less, more preferably 7 d or less, more preferably 5 d or less, and even more preferably 3.5 d or less. According to particularly preferred embodiments, the inventive process is provided with the proviso that for crystallization temperatures of 175° C. or higher in step (2) the duration thereof is less than 14 d, preferably with the proviso that for crystallization temperatures of 170° C. or higher in step (2) the duration thereof is 13 d or less, more preferably with the proviso that for crystallization temperatures of 165° C. or higher in step (2) the duration thereof is 7 d or less, more preferably with the proviso that for crystallization temperatures of 160° C. or higher in step (2) the duration thereof is 5 d or less, more preferably with the proviso that for crystallization temperatures of 155° C. or higher in step (2) the duration thereof is 3.5 d or less.

According to preferred embodiments of the present invention, wherein the mixture is heated in step (2), said heating may be conducted during the entire crystallization process or during only one or more portions thereof, provided that a zeolitic material having the LEV-type framework structure is crystallized. Preferably, heating is conducted during the entire duration of crystallization.

In general, the process of the present invention can optionally comprise further steps for the work-up and/or further physical and/or chemical transformation of the zeolitic material having an LEV-type framework structure crystallized in step (2) from the mixture provided in step (1). The crystallized material can for example be subject to any sequence of isolation (3) and/or washing procedures (4), wherein the zeolitic material obtained from crystallization in step (2) is preferably subject to at least one isolation (3) and at least one washing procedure (4).

Isolation of the crystallized product can be achieved by any conceivable means. Preferably, isolation of the crystallized product can be achieved by means of filtration, ultrafiltration, diafiltration, centrifugation and/or decantation methods, wherein filtration methods can involve suction and/or pressure filtration steps. Furthermore, for ease of isolation, any suitable compounds or compositions and in particular any suitable flocculating agent may be added and/or any suitable treatment may be performed to the zeolitic material obtained from crystallization in step (2) for facilitating the isolation procedure.

According to particularly preferred embodiments of the inventive process, after step (2) and prior to isolating (3) and/or washing (4) and/or drying (5) the zeolitic material having an LEV-type framework structure, preferably prior to isolating and/or washing, and even more preferably prior to isolating the zeolitic material, the pH of the crystallization product and in particular of the zeolitic material is adjusted to a pH in the range of from 5 to 12, preferably from 6 to 11, more preferably from 7 to 10, more preferably from 8 to 9.5, and even more preferably to a pH in the range of from 8.3 to 9.3. Thus, it has surprisingly been found that when adjusting the pH of the crystallization product and in particular of the zeolitic material having an LEV-type framework structure, the ease of isolating (3) and/or washing (4) the zeolitic material is greatly improved. In particular, the zeolitic material of which the pH has been adjusted accordingly may be easily filtered off and/or washed, and does not necessitate elaborate isolation and/or washing techniques involving for example ultrafiltration, diafiltration, and/or centrifugation, or combinations thereof, for isolation (3) and/or washing (4) thereof. These advantages have a particular impact on production methods conducted on a large scale and in particular on an industrial scale, since filtration means commonly used one this scale may be used in a highly efficient manner.

With respect to one or more optional washing procedures, any conceivable solvent can be used. Washing agents which may be used are, for example, water, alcohols, such as methanol, ethanol or propanol, or mixtures of two or more thereof. Examples of mixtures are mixtures of two or more alcohols, such as methanol and ethanol or methanol and propanol or ethanol and propanol or methanol and ethanol and propanol, or mixtures of water and at least one alcohol, such as water and methanol or water and ethanol or water and propanol or water and methanol and ethanol or water and methanol and propanol or water and ethanol and propanol or water and methanol and ethanol and propanol. Water or a mixture of water and at least one alcohol, preferably water and ethanol, is preferred, distilled water being very particularly preferred as the only washing agent.

Preferably, the separated zeolitic material is washed until the pH of the washing agent, preferably the washwater, is in the range of from 6 to 8, preferably from 6.5 to 7.5, as determined via a standard glass electrode. According to embodiments which are further preferred, the separated zeolitic material is washed until the washing solvent or solvent mixture which is preferably water and more preferably distilled water displays a conductivity of 1000 μS/cm³ or less, more preferably of 500 μS/cm³ or less, more preferably of 200 μS/cm³ or less, more preferably of 100 μS/cm³ or less, and even more preferably of 50 μS/cm³ or less. Furthermore, the inventive process can optionally comprise one or more drying steps (5). In general, any conceivable means of drying can be used. Drying procedures preferably include heating and/or applying vacuum to the zeolitic material having an LEV-type framework structure. In alternatively preferred embodiments of the present invention, one or more drying steps may involve spray drying, and preferably spray granulation of the zeolitic material.

In embodiments which comprise at least one drying step (5), the drying temperatures are preferably in the range of from 25° C. to 150° C., more preferably of from 60 to 140° C., more preferably of from 70 to 130° C. and even more preferably in the range of from 75 to 125° C. The durations of drying are preferably in the range of from 2 to 60 h, more preferably in the range of 6 to 48 hours, and even more preferably of from 12 to 24 h.

Furthermore, in addition to or alternatively to at least one drying step, the inventive process preferably includes a calcination procedure which is preferably conducted after a step of drying the zeolitic material having an LEV-type framework structure. In principle, there is no particular restriction according to the present invention as to the temperature used for the preferred calcinations step, nor with respect to the duration thereof. Thus, by way of example, the calcination may be performed at a temperature ranging anywhere from 300 to 900° C., wherein it preferred that the calcinations be performed at a temperature comprised in the range of from 400 to 800° C., more preferably from 500 to 700° C., and even more preferably from 550 to 650° C. Furthermore, by way of example, the calcination may suitably be performed for a duration of from 1 to 48 h, wherein calcination is preferably performed for a duration of from 2 to 36 h, more preferably of from 4 to 24 h, more preferably of from 6 to 20 h, more preferably of from 8 to 12 h, and even more preferably of from 9 to 11.

According to the inventive process, the zeolitic material crystallized in step (2) can optionally be subject to at least one step of an ion-exchange procedure (6), wherein the term “ion-exchange” according to the present invention generally refers to non-framework ionic elements and/or molecules contained in the zeolitic material. Preferably, the non-framework ionic element comprises one or more of the one or more alkali metals M optionally comprised in the zeolitic material have an LEV-type framework structure according to embodiments and preferred embodiments of the present invention, and is preferably sodium and/or potassium, more preferably sodium.

In general, any conceivable ion-exchange procedure with one or more suitable ionic elements and/or molecules can be conducted on the zeolitic material. Preferably, as ionic elements at least one cation and/or cationic element is employed which is preferably selected from the group consisting of H⁺, NH₄⁺, Sr, Zr, Cr, Fe, Co, Ni, Cu, Zn, Ru, Rh, Pd, Ag, Os, Ir, Pt, Au, and mixtures of two or more thereof, more preferably from the group consisting of H⁺, NH₄⁺, Sr, Cr, Fe, Co, Ni, Cu, and mixtures of two or more thereof, and even more preferably from the group consisting of H⁺, NH₄⁺, Fe, Cu, and mixtures of two or more thereof. Preferably, the zeolitic material is first ion-exchanged with H⁺ and/or NH₄⁺, and more preferably with NH₄⁺, before being subject to a further ion-exchange procedure, more preferably before being subject to ion-exchange with at least one cation and/or cationic element selected from the group consisting Sr, Zr, Cr, Fe, Co, Ni, Cu, Zn, Ru, Rh, Pd, Ag, Os, Ir, Pt, Au, and mixtures of two or more thereof, more preferably from the group consisting of Sr, Cr, Fe, Co, Ni, Cu, and mixtures of two or more thereof, wherein even more preferably the at least one cation and/or cationic element comprises or preferably consists of Cu and/or Fe.

In general, the preferred isolation (3) and/or washing (4) and/or drying and/or calcining (5) and/or ion-exchange procedures (6) comprised in the inventive process can be conducted the aforementioned sequence or in any conceivably order and combination and may be repeated as often as desired.

Therefore, the inventive process preferably further comprises one or more of the following steps of
(3) isolating the zeolitic material having an LEV-type framework structure, preferably by filtration, ultrafiltration, diafiltration, centrifugation and/or decantation methods, and/or
(4) washing the zeolitic material having an LEV-type framework structure, and/or
(5) drying and/or calcining the zeolitic material having an LEV-type framework structure, and/or
(6) subjecting the zeolitic material having an LEV-type framework structure to an ion-exchange procedure,
   wherein the steps (3) and/or (4) and/or (5) and/or (6) can be conducted in any order, and wherein one or more of said steps is preferably repeated one or more times.

Preferably, the inventive process comprises at least one step of isolating the zeolitic material crystallized according to step (2), more preferably by filtration thereof. According to said preferred embodiment it is further preferred that after the at least one step of isolating, the zeolitic material is subject to at least one step of drying, wherein more preferably the zeolitic material is subject to at least one step of washing prior to the at least one drying step. In a particularly preferred embodiment, the zeolitic material crystallized according to step (2) is subject to at least one step of isolating, followed by at least one step of washing, followed by at least one step of drying. According to particularly preferred embodiments, pH of the zeolitic material crystallized according to step (2) is additionally adjusted according to preferred embodiments of the inventive process prior to the at least one step of isolating and/or to the at least one step of washing, preferably prior to the at least one step of isolating.

According to an alternative embodiment of the inventive process which is preferred, the zeolitic material crystallized in step (2) is directly subject to at least one step of drying, preferably to spray drying and or spray granulation, preferably without isolating (3) and/or washing (4) and/or drying (5) of the zeolitic material beforehand. Directly subjecting the mixture obtained from step (2) of the inventive process to a spray drying or spray granulation stage has the advantage that isolation and drying is performed in a single stage. Consequently, according to this embodiment of the present invention, an even more preferred process is provided wherein the number of post-synthesis workup steps is further minimized, as a result of which the zeolitic material having an LEV-type framework structure can be obtained from a highly efficient process. According to or alternatively to said preferred embodiment involving a spray drying procedure directly after obtained the zeolitic material in step (2), it is further preferred to apply a step of spray drying to the zeolitic material instead of or directly following any one or more of: isolating (3) and/or washing (4) and/or drying and/or calcining (5) the zeolitic material and/or subjecting the zeolitic material to one or more ion-exchange procedures (6).

The present invention furthermore relates to a zeolitic material having an LEV-type framework structure which is either obtained by the process according to the present invention or by any conceivable process which leads to a zeolitic material having an LEV-type framework structure as obtainable according to the inventive process.

Therefore, the present invention also relates to a zeolitic material having an LEV-type framework structure which is obtainable and/or obtained according to the inventive process, and in particular which is obtainable and/or obtained according to any embodiment or preferred embodiment of the inventive process as outlined in the foregoing.

Furthermore, an embodiment of the present invention also relates to a zeolitic material as such having an LEV-type framework structure comprising YO₂ and X₂O₃, wherein Y is a tetravalent element, and X is a trivalent element, and wherein said zeolitic material optionally comprises one or more alkali metals M. Furthermore, according to an embodiment of the present invention, the zeolitic material display a Y:X atomic ratio comprised in the range of from 1 to 9.4, and preferably of from 2 to 9.3, more preferably of from 3 to 9.1, more preferably of from 5 to 9.0, more preferably of from 7.5 to 8.9, more preferably of from 8 to 8.8, wherein even more preferably a Y:X atomic ratio is comprised in the range of from 8.5 to 8.7.

Therefore, an embodiment of the present invention is also directed to a zeolitic material having an LEV-type framework structure, preferably obtainable and/or obtained according to any one of the embodiments and preferred embodiments of the inventive process, said zeolitic material comprising YO₂ and X₂O₃,

wherein Y is a tetravalent element, and X is a trivalent element,
wherein the zeolitic material optionally comprises one or more alkali metals M, preferably sodium and/or potassium, and more preferably sodium, and
wherein the zeolitic material displays an Y:X atomic ratio of from 1 to 9.4, preferably from 2 to 9.3, more preferably from 3 to 9.1, more preferably from 5 to 9.0, more preferably from 7.5 to 8.9, more preferably from 8 to 8.8, and even more preferably from 8.5 to 8.7.

According to further preferred embodiments, Y is selected from the group consisting of Si, Sn, Ti, Zr, Ge, and mixtures of two or more thereof, Y preferably being Si. Furthermore, in preferred embodiments comprising X₂O₃, it is further preferred that X is selected from the group consisting of Al, B, In, Ga, and mixtures of two or more thereof, X preferably being Al and/or B, and more preferably being Al.

Thus, it has surprisingly been found that according to an embodiment of the present invention a zeolitic material having an LEV-type framework structure comprising YO₂ and X₂O₃ may be provided displaying exceptionally low Y:X atomic ratios and, according to particularly preferred embodiments wherein Y comprises Si and X comprises Al, exceptionally low Si:Al ratios. This unexpected finding is highly advantageous since in zeolitic materials having an LEV-type framework structure comprising YO₂ and X₂O₃, in general the X element provides active sites for a variety of applications including their use as a molecular sieve or adsorbent and catalytic applications wherein the zeolitic material may either be provided as the catalytically active component and/or as the catalyst support. Accordingly, due to the exceptionally low Y:X ratios and, according to the aforementioned particularly preferred embodiments comprising Si as Y and Al as X, due to exceptionally low Si:Al ratios in said particularly preferred embodiments, a highly efficient zeolitic material having an LEV-type framework structure may be provided, in particular due to the higher density of active sites provided by the framework element X and, in particular, by Al comprised in the zeolitic framework. The advantages of such highly active zeolitic materials are numerous, in particular with respect to the possibility of achieving the same effects as conventional zeolitic materials having higher Y:X ratios using far less material. In addition to this, the higher density of the active sites and in particular of catalytically active sites in particular applications, leads to novel chemical activities and applications which may not be provided by conventional zeolitic materials.

According to preferred embodiments of the present invention, the zeolitic material having an LEV-type framework structure has an X-ray diffraction pattern comprising at least the following reflections:

| Intensity (%) | Diffraction angle 2θ/° [Cu K(alpha 1)] |
|---|---|
| [26-40] | [10.94-11.20] |
| [23-27] | [13.36-13.62] |
| [50-66] | [17.33-17.62] |
| [42-57] | [21.05-21.37] |
| 100 | [22.08-22.21] |
| [35-56] | [28.59-28.92] |
| [48-62] | [32.37-32.60] |
| [12-19] | [51.60-52.14] |
| [11-13] | [55.68-56.32] |

wherein 100% relates to the intensity of the maximum peak in the X-ray powder diffraction pattern.

Preferably, the zeolitic material having an LEV-type framework structure has an X-ray diffraction pattern comprises at least the following reflections:

| Intensity (%) | Diffraction angle 2θ/° [Cu K(alpha 1)] |
|---|---|
| [29-37] | [10.99-11.15] |
| [24-27] | [13.41-13.56] |
| [53-63] | [17.39-17.56] |
| [45-54] | [21.11-21.31] |
| [100] | [22.11-22.18] |
| [39-52] | [28.66-28.86] |
| [51-59] | [32.42-32.56] |
| [13-18] | [51.71-52.03] |
| [11-13] | [55.81-56.19] |

wherein 100% relates to the intensity of the maximum peak in the X-ray powder diffraction pattern.

According to preferred embodiments, at least a portion of the alkali metals M optionally present in the zeolitic material having an LEV-type framework structure are substituted by one or more cation and/or cationic element. In general, any suitable cation and/or cationic element may substitute the alkali metals M, provided that an ion-exchanged zeolitic material having an LEV-type framework structure may be provided. According to an embodiment of the present invention it is preferred that the one or more cation and/or cationic element is selected from the group consisting of H⁺, NH₄⁺, Sr, Zr, Cr, Fe, Co, Ni, Cu, Zn, Ru, Rh, Pd, Ag, Os, Ir, Pt, Au, and mixtures of two or more thereof, and more preferably from the group consisting of H⁺, NH₄⁺, Sr, Cr, Fe, Co, Ni, Cu, and mixtures of two or more thereof. According to particularly preferred embodiments, the one or more cation and/or cationic element is selected from the group consisting of H⁺, NH₄⁺, Fe, Cu, and mixtures of two or more thereof, wherein even more preferably the one or more cation comprises Cu and/or Fe, Cu being particularly preferred.

According to embodiments of the present invention which are further preferred, at least a portion of the Y atoms in the LEV-type framework is isomorphously substituted by one or more element. In general, Y can be isomorphously substituted by any suitable element, wherein the one or more element is preferably selected from the group consisting of B, Fe, Ti, Sn, Ga, Ge, Zr, V, Nb, Cu, Zn, Li, Be, and mixtures of two or more thereof, more preferably wherein the one or more elements are selected from the group consisting of B, Fe, Ti, Sn, Zr, Cu, and mixtures of two or more thereof, wherein even more preferably the one or more element is Ti and/or B, preferably Ti. According to further embodiments of the present invention which are particularly preferred, the one or more element comprises Cu, wherein it is particularly preferred that the one or more element is Cu. According to yet further embodiments of the present invention which are particularly preferred, the one or more element comprises Fe, wherein it is particularly preferred that the one or more element is Fe.

In general, there is no particular restriction according to the present invention as to the portion of the Y atoms which may be isomorphously substituted in the LEV-type framework of the preferred zeolitic material. According to preferred embodiments, the molar ratio of YO₂ to the one or more element ranges from 5 to 100, preferably from 10 to 80, more preferably from 20 to 70, and even more preferably from 25 to 65

According to preferred embodiments of the present invention, the zeolitic material having an LEV-type framework structure, and preferably the calcined zeolitic material, has a BET surface area determined according to DIN 66135 which is comprised in the range of from 50 to 1000 m²/g, and preferably of from 200 to 950 m²/g, more preferably of from 500 to 900 m²/g, more preferably of from 600 to 850 m²/g, and even more preferably of from 650 to 800 m²/g.

Furthermore, as described above with respect to the inventive process, also the zeolitic material according to the present invention may comprise one or more of any conceivable zeolites having an LEV-type framework structure, provided that said one or more zeolites contain YO₂ and X₂O₃ wherein YO₂ and X₂O₃ are preferably at least in part contained as a framework element therein, respectively. According to preferred embodiments of the present invention, the zeolitic material comprises one or more zeolites selected from the group consisting of Levyne, LZ-132, NU-3, RU B-1, ZK-20, ZSM-45, RUB-50, and mixtures of two or more thereof, wherein it is particularly preferred that the zeolitic material comprises RUB-50.

Depending on the specific needs of its application, the inventive material can be employed as such, like in the form of a powder, a spray powder or a spray granulate obtained from above-described separation techniques, e.g. decantation, filtration, centrifugation, or spraying.

In many industrial applications, it is often desired on the part of the user not to employ the zeolitic material as powder or sprayed material, i.e. the zeolitic material obtained by the separation of the material from its mother liquor, optionally including washing and drying, and subsequent calcination, but a zeolitic material which is further processed to give moldings. Such moldings are required particularly in many industrial processes, e.g. in many processes wherein the zeolitic material of the present invention is employed as catalyst or adsorbent.

Accordingly, the present invention also relates to the zeolitic material of the present invention having an LEV-type framework structure which is comprised in a molding.

In general, the powder or sprayed material can be shaped without any other compounds, e.g. by suitable compacting, to obtain moldings of a desired geometry, e.g. tablets, cylinders, spheres, or the like.

Preferably, the powder or sprayed material is admixed with or coated by a suitable refractory binder. In general, suitable binders are all compounds which impart adhesion and/or cohesion between the zeolitic material particles to be bonded which goes beyond the physisorption which may be present without a binder. Examples of such binders are metal oxides, such as, for example, SiO₂, Al₂O₃, TiO₂, ZrO₂ or MgO or clays, or mixtures of two or more of these compounds. Naturally occurring clays which can be employed include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or initially subjected to calcination, acid treatment or chemical modification. In addition, the zeolitic material according to the present invention can be composited with a porous matrix material such as silica-alumina, silica-magnesia, silica-zirconia, silica-thoria, silica-beryllia and silica-titania as well as ternary compositions such as silica-alumina-thoria, silica-alumina-zirconia, silica-alumina-magnesia and silica-magnesia-zirconia.

The moldings of an embodiment of the present invention may therefore also be provided in the form of extrudates, pellets, tablets or particles of any other suitable shape, for use as a packed bed of particulate catalyst, or as shaped pieces such as plates, saddles, tubes, or the like.

Also preferably, the powder or the sprayed material, optionally after admixing or coating by a suitable refractory binder as described above, is formed into a slurry, for example with water, which is deposited upon a suitable refractory carrier. The slurry may also comprise other compounds such as, e.g., stabilizers, defoamers, promotors, or the like. Typically, the carrier comprises a member, often referred to as a “honeycomb” carrier, comprising one or more refractory bodies having a plurality of fine, parallel gas flow passages extending therethrough. Such carriers are well known in the art and may be made of any suitable material such as cordierite or the like.

In general, the zeolitic material described above can be used as molecular sieve, adsorbent, catalyst, catalyst support or binder thereof. For example, the zeolitic material can be used as molecular sieve to dry gases or liquids, for selective molecular separation, e.g. for the separation of hydrocarbons or amides; as ion exchanger; as chemical carrier; as adsorbent, in particular as adsorbent for the separation of hydrocarbons or amides; or as a catalyst. Most preferably, the zeolitic material according to the present invention is used as a catalyst and/or as a catalyst support.

According to a preferred embodiment of the present invention, the zeolitic material of the invention is used in a catalytic process, preferably as a catalyst and/or catalyst support, and more preferably as a catalyst. In general, the zeolitic material of the invention can be used as a catalyst and/or catalyst support in any conceivable catalytic process, wherein processes involving the conversion of at least one organic compound is preferred, more preferably of organic compounds comprising at least one carbon-carbon and/or carbon-oxygen and/or carbon-nitrogen bond, more preferably of organic compounds comprising at least one carbon—carbon and/or carbon-oxygen bond, and even more preferably of organic compounds comprising at least one carbon-carbon bond. In particularly preferred embodiments of the present invention, the zeolitic material is used as a catalyst and/or catalyst support in any one or more of methanol-to-olefin (MTO) reactions, ethylene-to-propylene (ETP) reactions, as well as of the co-reaction of methanol and ethylene (CME).

According to a further embodiment of the present invention, the zeolitic material of the invention is preferably used in a catalytic process involving the conversion of at least one compound comprising at least one nitrogen-oxygen bond. Particularly preferred according to the present invention is the use of the zeolitic material having an LEV-type framework structure as a catalyst and/or catalyst support in a selective catalytic reduction (SCR) process for the selective reduction of nitrogen oxides NO_{R}; for the oxidation of NH₃, in particular for the oxidation of NH₃ slip in diesel systems; for the decomposition of N₂O. The term nitrogen oxides, NOₓ, as used in the context of the present invention designates the oxides of nitrogen, especially dinitrogen oxide (N₂O), nitrogen monoxide (NO), dinitrogen trioxide (N₂O₃), nitrogen dioxide (NO₂), dinitrogen tetroxide (N₂O₄), dinitrogen pentoxide (N₂O₅), nitrogen peroxide (NO₃). According to particularly preferred embodiments of the present invention, the zeolitic material used in a catalytic process involving the conversion of at least one compound comprising at least one nitrogen-oxygen bond comprises Cu and/or Fe, and more preferably Cu.

Therefore, the present invention also relates to a method for selectively reducing nitrogen oxides NOₓ by contacting a stream containing NOₓ with a catalyst containing the zeolitic material having an LEV-type framework structure according to the present invention under suitable reducing conditions; to a method of oxidizing NH₃, in particular of oxidizing NH₃ slip in diesel systems, by contacting a stream containing NH₃ with a catalyst containing the zeolitic material having an LEV-type framework structure according to the present invention under suitable oxidizing conditions; to a method of decomposing of N₂O by contacting a stream containing N₂O with a catalyst containing the zeolitic material having an LEV-type framework structure according to the present invention under suitable decomposition conditions; to a method of controlling emissions in Advanced Emission Systems such as Homogeneous Charge Compression Ignition (HCCl) engines by contacting an emission stream with a catalyst containing the zeolitic material having an LEV-type framework structure according to the present invention under suitable conditions; to a fluid catalytic cracking FCC process wherein the zeolitic material having an LEV-type framework structure according to the present invention is employed as additive; to a method of converting an organic compound by contacting said compound with a catalyst containing the zeolitic material having an LEV-type framework structure according to the present invention under suitable conversion conditions; to a “stationary source” process wherein a catalyst is employed containing the zeolitic material having an LEV-type framework structure according to the present invention.

Accordingly, the present invention also relates to a method for selectively reducing nitrogen oxides NOₓ, wherein a gaseous stream containing nitrogen oxides NOₓ, preferably also containing ammonia and/urea, is contacted with the zeolitic material according to the present invention or the zeolitic material obtainable or obtained according to the present invention, preferably in the form of a molded catalyst, still more preferably as a molded catalyst wherein the zeolitic material is deposited on a suitable refractory carrier, still more preferably on a “honeycomb” carrier.

The nitrogen oxides which are reduced using a catalyst containing the zeolitic material according to the present invention or the zeolitic material obtainable of obtained according to the present invention may be obtained by any process, e.g. as a waste gas stream. Among others, waste gas streams as obtained in processes for producing adipic acid, nitric acid, hydroxylamine derivatives, caprolactame, glyoxal, methyl-glyoxal, glyoxylic acid or in processes for burning nitrogeneous materials may be mentioned.

Most preferably, the zeolitic material according to an embodiment of the present invention or the zeolitic material obtainable of obtained according to the present invention is used as a molded catalyst, still more preferably as a molded catalyst wherein the zeolitic material is deposited on a suitable refractory carrier, still more preferably on a “honeycomb” carrier, for the selective reduction of nitrogen oxides NOₓ, i.e. for selective catalytic reduction of nitrogen oxides. In particular, the selective reduction of nitrogen oxides wherein the zeolitic material according to an embodiment of the present invention is employed as catalytically active material is carried out in the presence ammonia or urea. While ammonia is the reducing agent of choice for stationary power plants, urea is the reducing agent of choice for mobile SCR systems. Typically, the SCR system is integrated in the engine and vehicle design and, also typically, contains the following main components:SCR catalyst containing the zeolitic material according to an embodiment of the present invention; a urea storage tank; a urea pump; a urea dosing system; a urea injector/nozzle; and a respective control unit.

Especially preferred is the use of a catalyst containing the zeolitic material according to the present invention or the zeolitic material obtainable or obtained according to the inventive process for removal of nitrogen oxides NO from exhaust gases of internal combustion engines, in particular diesel engines, which operate at combustion conditions with air in excess of that required for stoichiometric combustion, i.e. in a lean operation mode.

Therefore, the present invention also relates to a method for removing nitrogen oxides NO from exhaust gases of internal combustion engines, in particular diesel engines, which operate at combustion conditions with air in excess of that required for stoichiometric combustion, i.e., at lean conditions, wherein a catalyst containing the zeolitic material according to the present invention or the zeolitic material obtainable or obtained according to the present invention is employed as catalytically active material.

The present invention therefore relates to the use of the zeolitic material of the invention, in particular in the field of catalysis and/or in the treatment of exhaust gas, wherein said exhaust gas treatment comprises industrial and automotive exhaust gas treatment. In these and other applications, the zeolitic material of the present invention can by way of example be used as a molecular sieve, catalyst, and/or catalyst support.

Furthermore, it is preferred according to the present invention that the zeolitic material is used as a molecular trap for organic compounds. In general, any type of organic compound may be trapped in the zeolitic material, wherein it is preferred that the compound is reversibly trapped, such that it may be later released from the zeolitic material, preferably wherein the organic compound is released—preferably without conversion thereof—by an increase in temperature and/or a decrease in pressure. Furthermore, it is preferred that the zeolitic material is used to trap organic compounds of which the dimensions allow them to penetrate the microporous system of the molecular structure. According to yet further embodiments of the present invention, it is preferred that the trapped compounds are released under at least partial conversion thereof to a chemical derivative and/or to a decomposition product thereof, preferably to a thermal decomposition product thereof.

Therefore, an embodiment of the present invention also relates to the use of a zeolitic material according to embodiments and preferred embodiments of the present invention as a molecular sieve, catalyst, catalyst support, and/or as an adsorbent, wherein the zeolitic material is preferably used as a molecular trap for chemical compounds, as a catalyst and/or as a catalyst support.

When preparing specific catalytic compositions or compositions for different purposes, it is also conceivable to blend the zeolitic material according to the present invention having an LEV-type framework structure with at least one other catalytically active material or a material being active with respect to the intended purpose. It is also possible to blend at least two different inventive materials which may differ in the Y:X ratio, preferably in the Si:Al ratio, and/or in the presence or absence of one or more further metals such as one or more transition metals and/or in the specific amounts of a further metal such as a transition metal, wherein according to particularly preferred embodiments, the one or more transition metal comprises Cu and/or Fe, more preferably Cu. It is also possible to blend at least two different inventive materials with at least one other catalytically active material or a material being active with respect to the intended purpose.

As mentioned in the foregoing with respect to preferred embodiments, the zeolitic material having an LEV-type framework structure may be disposed on a substrate. In general, the substrate may be any of those materials typically used for preparing catalysts, and preferably comprises a ceramic or metal honeycomb structure. Any suitable substrate may be employed, such as a monolithic substrate of the type having fine, parallel gas flow passages extending therethrough from an inlet or an outlet face of the substrate, such that passages are open to fluid flow therethrough (referred to as honeycomb flow through substrates). The passages, which are essentially straight paths from their fluid inlet to their fluid outlet, are defined by walls on which the zeolitic material is disposed as a washcoat so that the gases flowing through the passages contact the catalytic material. The flow passages of the monolithic substrate are thin-walled channels, which can be of any suitable cross-sectional shape and size such as trapezoidal, rectangular, square, sinusoidal, hexagonal, oval, circular, etc. Such structures may contain from about 60 to about 400 or more gas inlet openings (i.e., cells) per square inch (2.54 cm×2.54 cm) of cross section.

The substrate can also be a wall-flow filter substrate, where the channels are alternately blocked, allowing a gaseous stream entering the channels from one direction (inlet direction), to flow through the channel walls and exit from the channels from the other direction (outlet direction). The catalyst composition can be coated on the flow through or wall-flow filter. If a wall flow substrate is utilized, the resulting system will be able to remove particulate matter along with gaseous pollutants. The wall-flow filter substrate can be made from materials commonly known in the art, such as cordierite, aluminum titanate or silicon carbide. It will be understood that the loading of the catalytic composition on a wall flow substrate will depend on substrate properties such as porosity and wall thickness, and typically will be lower than loading on a flow through substrate.

The ceramic substrate may be made of any suitable refractory material, e.g., cordierite, cordierite-alumina, silicon nitride, zircon mullite, spodumene, alumina-silica magnesia, zircon silicate, sillimanite, a magnesium silicate, zircon, petalite, alpha-alumina, an aluminosilicate, and the like.

The substrates useful for embodiments of the present invention wherein the inventive zeolitic material is used as a catalyst may also be metallic in nature and be composed of one or more metals or metal alloys. The metallic substrates may be employed in various shapes such as corrugated sheet or monolithic form. Suitable metallic supports include the heat resistant metals and metal alloys such as titanium and stainless steel as well as other alloys in which iron is a substantial or major component. Such alloys may contain one or more of nickel, chromium and/or aluminum, and the total amount of these metals may advantageously comprise at least 15 wt. % of the alloy, e.g., 10-25 wt. % of chromium, 3-8 wt. % of aluminum and up to 20 wt. % of nickel. The alloys may also contain small or trace amounts of one or more other metals such as manganese, copper, vanadium, titanium, and the like. The surface or the metal substrates may be oxidized at high temperatures, e.g., 1000° C. and higher, to improve the resistance to corrosion of the alloys by forming an oxide layer on the surfaces of the substrates. Such high temperature-induced oxidation may enhance the adherence of the refractory metal oxide support and catalytically promoting metal components to the substrate.

In alternative embodiments, the zeolitic material according to the present invention having an LEV-type framework structure may be deposited on an open cell foam substrate. Such substrates are well known in the art, and are typically formed of refractory ceramic or metallic materials.

The powder X-ray diffraction patterns displayed in the figures were recorded on a Siemens D-5000 with monochromatic Cu K alpha-1 radiation, a capillary sample holder being used in order to avoid a preferred orientation. The diffraction data were collected using a position-sensitive detector from Braun, in the range from 8 to 96° (2 theta) and with a step width of 0.0678°. Indexing of the powder diagram was effected using the program Treor90, implemented in powder-X (Treor90 is a public domain program which is freely accessible via the URL http://www.ch.iucr.org/sincris-top/logiciel/). In the figure, the angle 2 theta in ° is shown along the abscissa and the intensities are plotted along the ordinate.

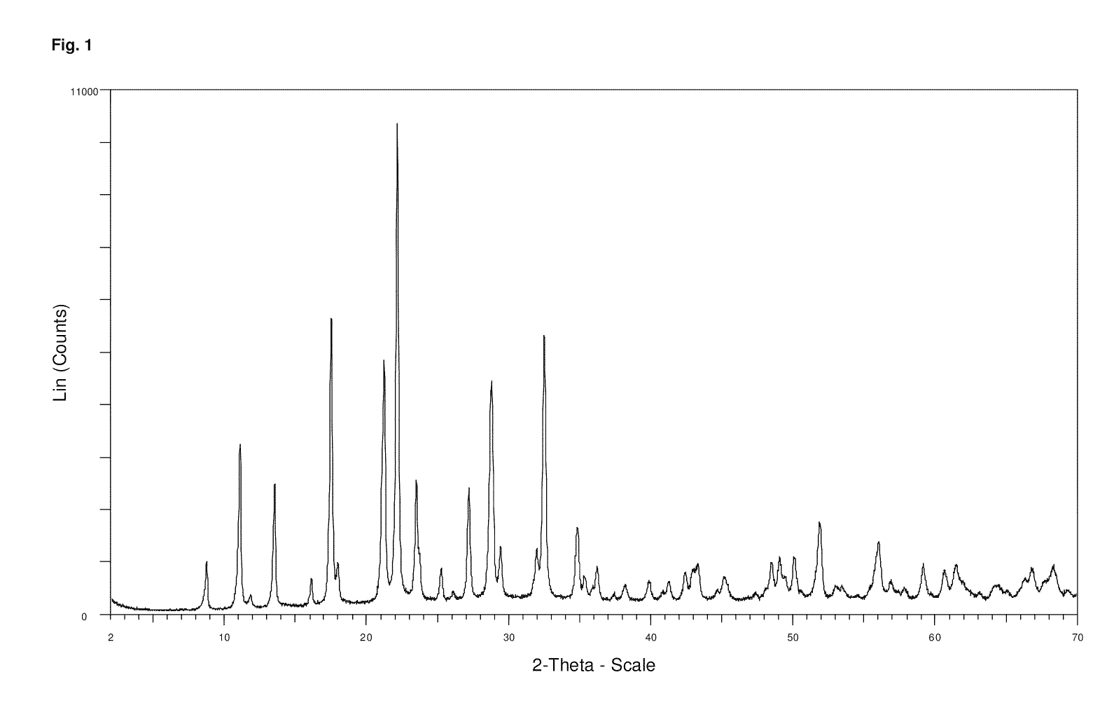

FIGS. 1, 2, 3B, and 4A show the X-ray diffraction pattern of the crystalline material obtained according to Examples 1, 2, 3, and 4, respectively.

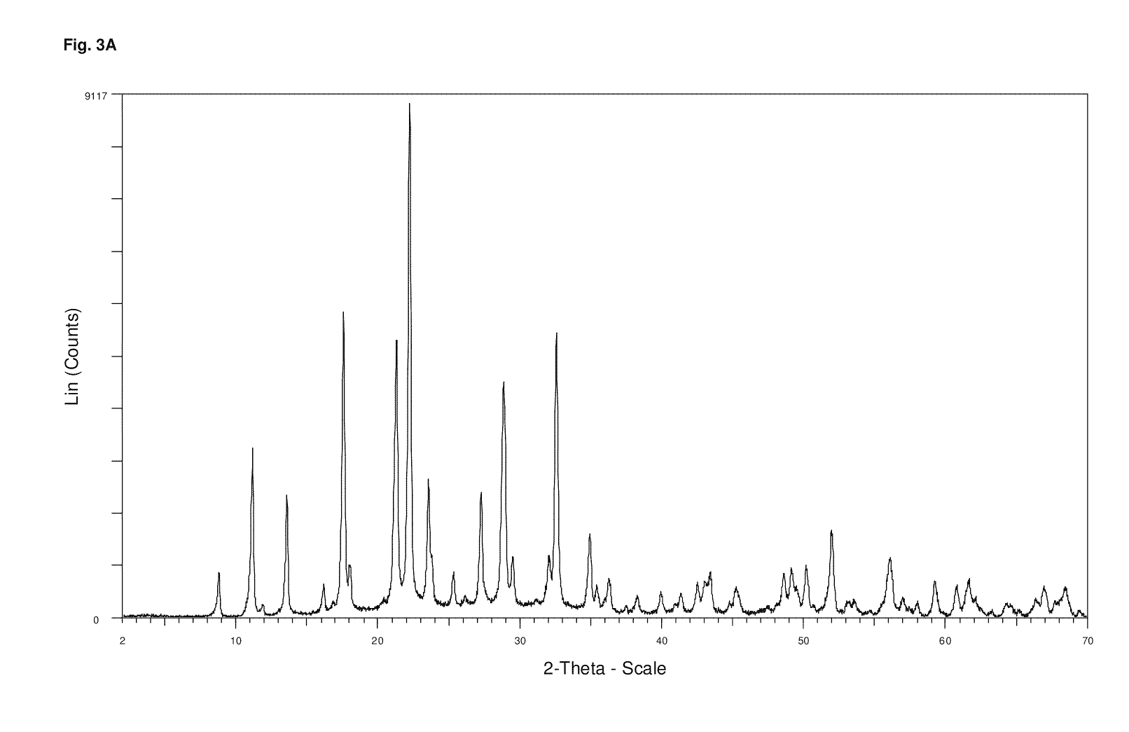

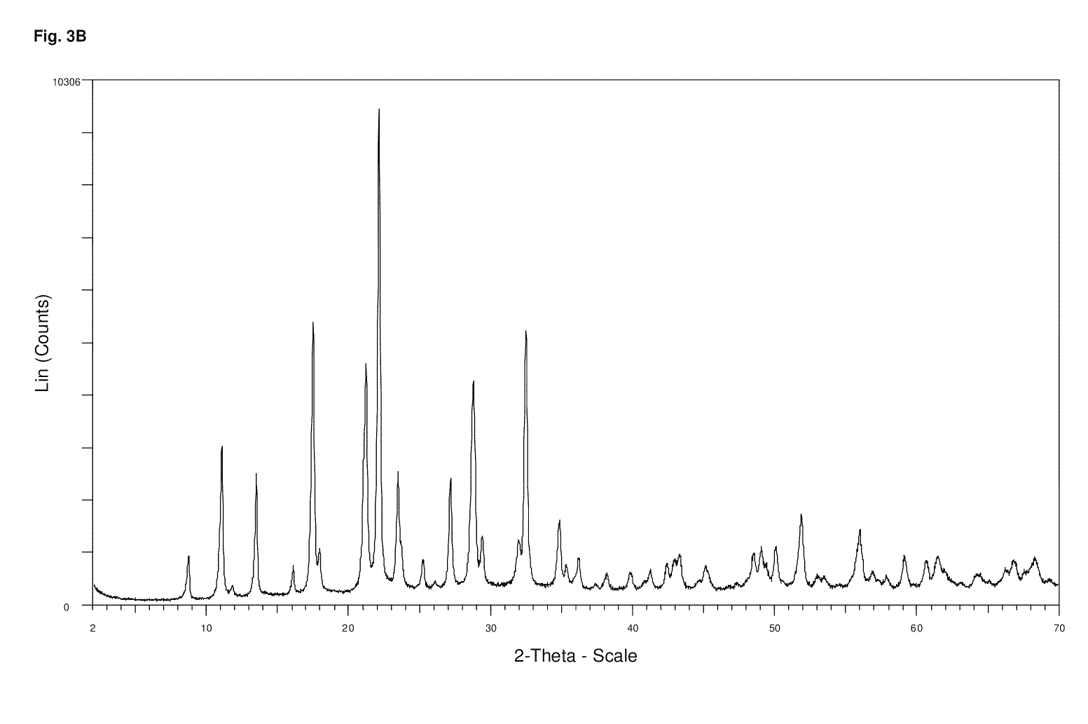

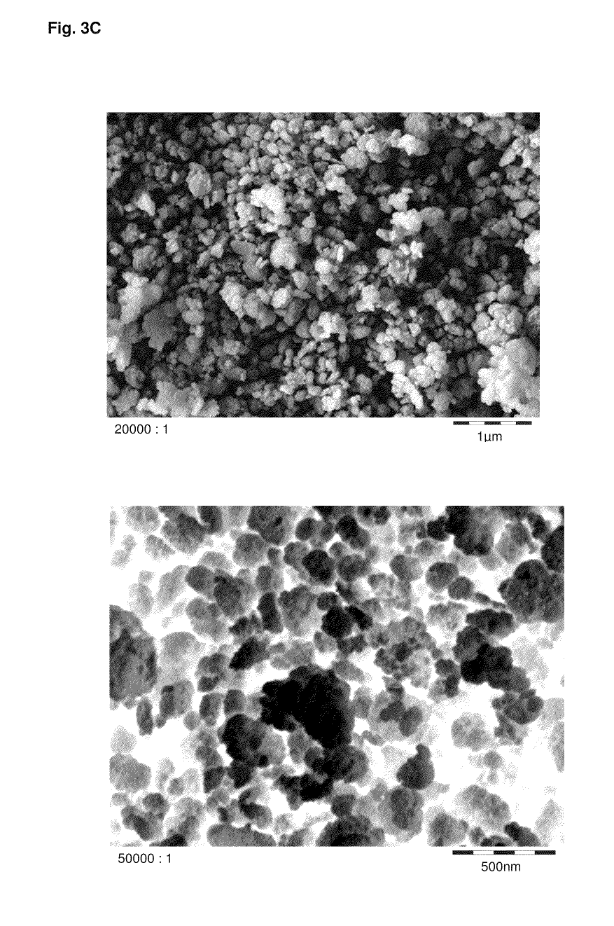

FIG. 3A shows the X-ray diffraction pattern of the RUB-50 seed crystals used in Example 3.

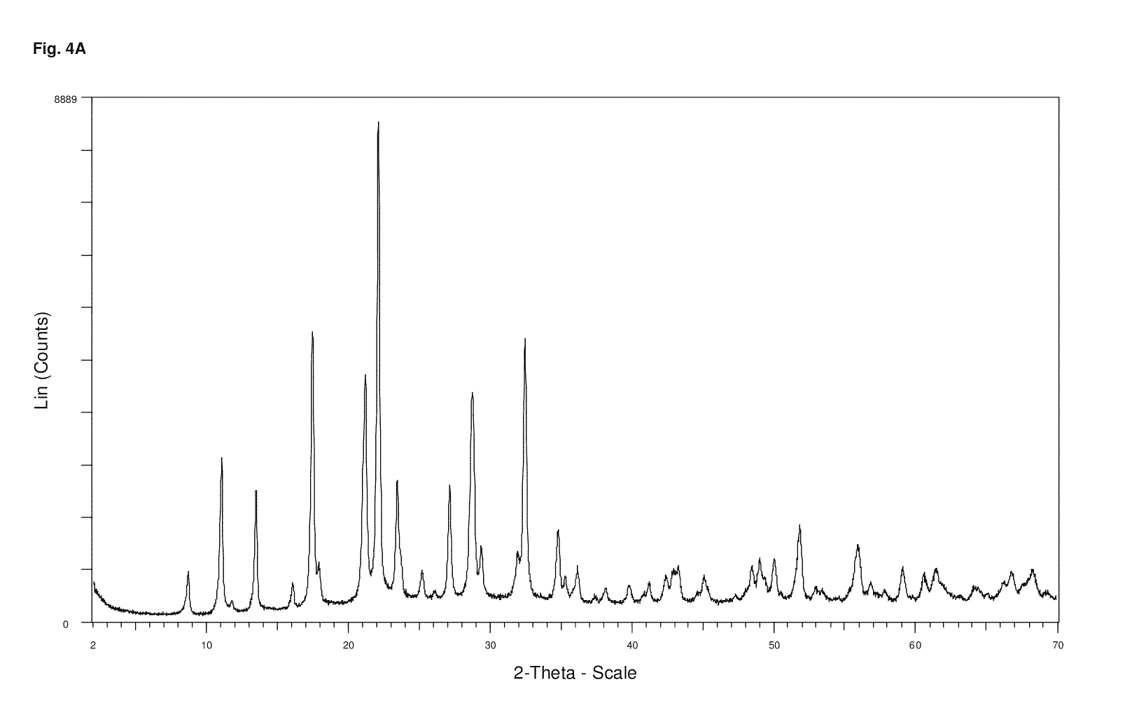

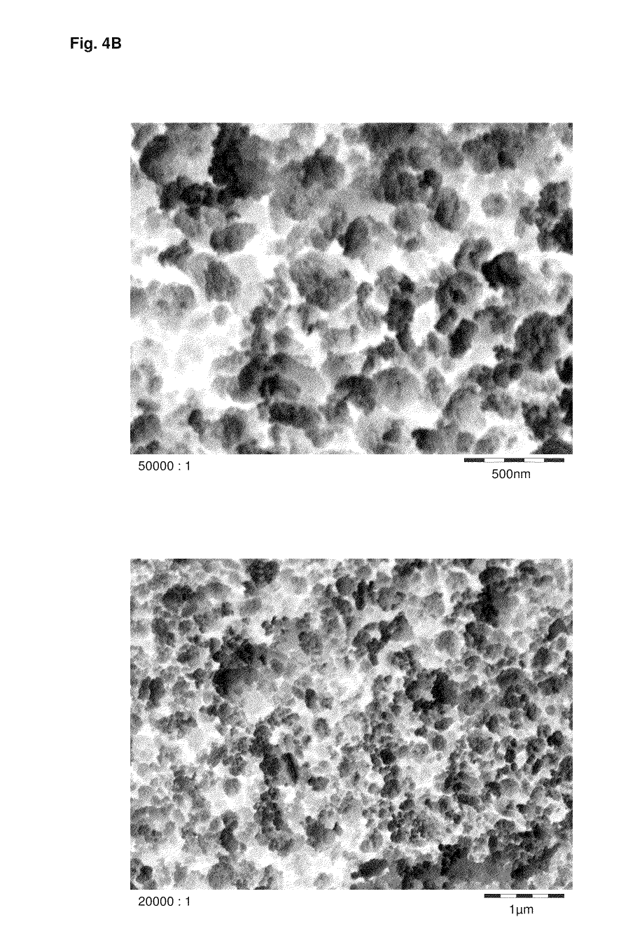

FIGS. 3C and 4B show the scanning electron microscope (SEM) images obtained from samples of the crystalline products obtained according to Examples 3 and 4, respectively.

### EXAMPLES

### Example 1

13.09 kg of an aqueous diethyldimethylammonium hydroxide solution (20.62 wt.-%) were placed in a plastic receptacle and 91.4 g of NaOH were then dissolved therein under stirring. 356.7 g of aluminum hydroxide were then added, and the resulting solution was then stirred for 10 min, to afford a slightly cloudy solution. 2.74 kg of fumed silica (Aerosil 200) were then added in portions and the resulting mixture stirred for 1 h to afford a milky suspension.

The mixture was then transferred to an autoclave and 6.3 kg of water was distilled off at 105° C., after which the resulting mixture was then crystallized under autogenous pressure at 150° C. for 120 hours (5 days). After having let the reaction mixture cool to room temperature, the reaction product consisted of a solid crust in a top portion, under which a white suspension was contained. The white suspension was isolated from the reaction mixture, centrifuged, and the white solid repeatedly elutriated with distilled water until a conductivity of the washing water of less than 200 μS/cm³ was achieved. The resulting wet cake was then dried at 120° C. for 16 h to afford 807.6 g of a white powder.

Elemental Analysis:
Si: 33 g/100 g
Al: 3.3 g/100 g
Na: 0.50 g/100 g

Therefore, according to elemental analysis, the Si:Al:Na molar ratio of the product is approximately 9.6:1:0.18.

FIG. 1 shows the XRD of the crystalline product obtained from the synthesis of Example 1. In particular, the XRD reflection pattern of the microcrystalline product reveals an LEV-type zeolite framework structure.

### Example 2

The procedure of Example 1 was repeated, wherein after completion of the hydrothermal synthesis, 501.3 g of the white suspension was isolated from the reaction product. The suspension, which displayed a pH-value of 12.8, was treated with 212 g of aqueous HNO₃ (10 wt.-%) to afford a pH of 6.5, after which 24.2 g of the white suspension was further added to afford a pH of 8.3. The solid was then filtered off and washed with 5.5 l of distilled water. The resulting solid was dried at 120° C. for 16 h thus affording 85.2 g of a white powder.

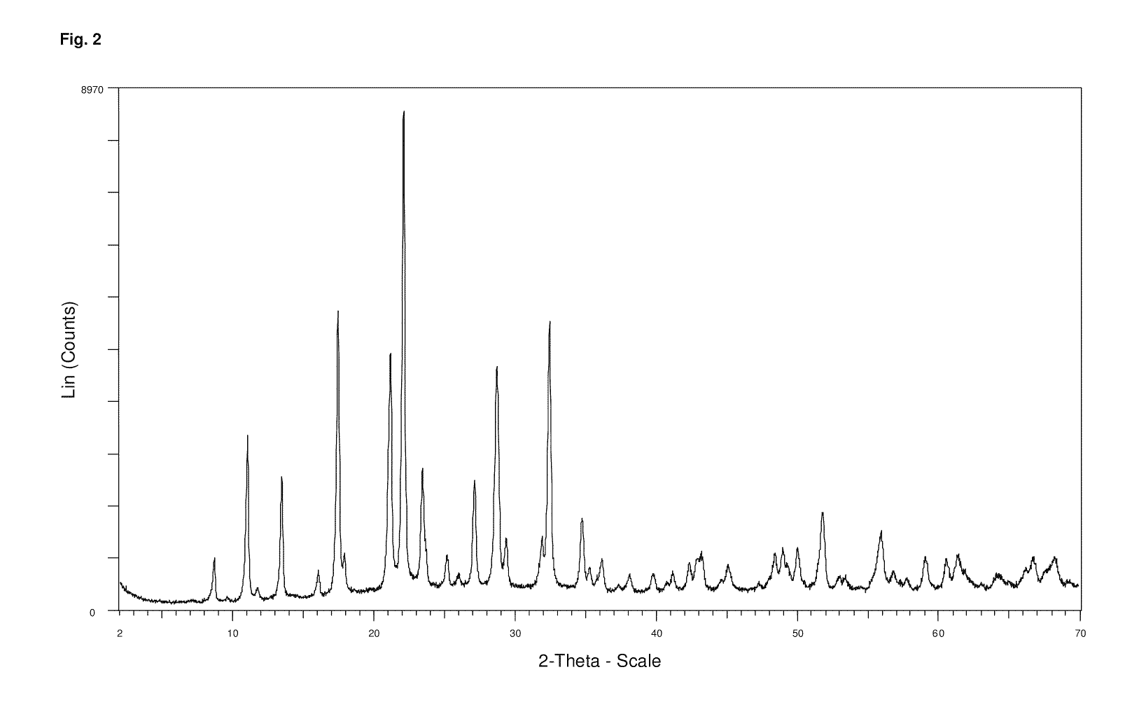

FIG. 2 shows the XRD of the crystalline product obtained from the synthesis of Example 2. In particular, the XRD reflection pattern of the microcrystalline product reveals an LEV-type zeolite framework structure.

Thus, compared to the procedure of Example 1, the additional step of acidification in Example 2 leads to a product which may be easily filtered and washed and which does not necessitate repeated steps of centrifugation and elutriation. This is in particular advantageous with respect to the large scale application of the inventive process.

### Example 3

### Preparation of the RUB-50 Seed Crystals

13.35 kg of an aqueous solution containing 20 wt.-% diethyldimethylammonium hydroxide were weighed into a plastic container after which 252.3 g of aluminum hydroxide were added under stirring and the solution was further stirred an additional 10 min to afford a slightly cloudy solution. 2.7 kg of Aerosil 200 were then added in portions under stirring after which the resulting suspension was stirred an additional 1 hour.

The mixture was then transferred to an autoclave wherein 6.6 kg of water were distilled off at a temperature of 105° C. The concentrated mixture was then crystallized under hydrothermal conditions at 150° C. for 246 hours (11 days), to afford 8.038 kg of an aqueous suspension.

The resulting suspension displayed a pH of 12.6 and was treated with 5.138 kg of aqueous HNO₃ (10 wt.-%) to afford a suspension with a pH of 8.2. The resulting solid was then filtered and washed with 40 l of distilled water to afford 7.52 kg of a wet product cake. Said cake was then transferred to a porcelain receptacle and dried at 120° C. for 16 hours, thus affording 2.365 kg of a white powder.

Elemental Analysis:

Si: 34 g/100 g

Al: 2.5 g/100 g

Therefore, according to the elemental analysis, the Si:Al ratio of the product is approximately 13.1.

In FIG. 3A, the XRD of the RUB-50 seed material is displayed, having the LEV-type zeolite framework structure.

### Seed-Directed Synthesis of an LEV-Type Zeolite

12.92 kg of an aqueous diethyldimethylammonium hydroxide solution (20.62 wt.-%) were placed in a plastic receptacle and 90.1 g of NaOH were then dissolved therein under stirring. 351.9 g of aluminum hydroxide were then added, and the resulting solution was then stirred for 10 min, to afford a slightly cloudy solution. 2.74 kg of fumed silica (Aerosil 200) were then added in portions and the resulting mixture stirred for 1 h, after which 135.3 g of the RUB-50 seed crystals were added and the mixture stirred a further 1 h to afford a milky suspension.

The mixture was then transferred to an autoclave and 6.2 kg of water was distilled off at 105° C., after which the resulting mixture was then crystallized under autogenous pressure at 150° C. for 168 hours (7 days). After having let the reaction mixture cool to room temperature, the reaction product consisted of a solid crust in a top portion, under which a white suspension was contained. The white suspension was isolated from the reaction mixture, centrifuged, and the white solid washed with distilled water until a conductivity of the washing water of less than 200 μS/cm³ was achieved. The resulting wet cake was then dried at 120° C. for 16 h to afford 1029.5 g of a white powder having a crystallinity grade of 94%, wherein the microcrystalline product displayed a mean diameter of the crystallites of 56.0 nm.

Electron Probe Micro Analysis of the crystalline product of Example 3 via Energy Dispersive X-Ray Spectroscopy (EDXS) afforded an Si:Al:Na molar ratio of about 11.4:1:0.3.

FIG. 3B shows the XRD of the crystalline product obtained from the synthesis of Example 1. In particular, the XRD reflection pattern of the microcrystalline product reveals an LEV-type zeolite framework structure.

FIG. 3C shows SEM-images of the crystalline product obtained according to Example 3.

### Example 4

The procedure of Example 3 was repeated, wherein after completion of the hydrothermal synthesis, 5698.4 g of the white suspension was isolated from the reaction product. The suspension, which displayed a pH-value of 12.35, was treated with 2996.6 g of aqueous HNO₃ (10 wt.-%) to afford a pH of 9.3. The solid was then filtered off and washed with 59 l of distilled water until a conductivity of the washing water of less than 200 μS/cm³ was achieved. The resulting wet cake was dried at 120° C. for 16 h thus affording 1561.6 g of a white powder.

Electron Probe Micro Analysis of the crystalline product of Example 4 via Energy Dispersive X-Ray Spectroscopy (EDXS) afforded an Si:Al:Na molar ratio of about 13:1:0.2.

FIG. 4A shows the XRD of the crystalline product obtained from the synthesis of Example 1. In particular, the XRD reflection pattern of the microcrystalline product reveals an LEV-type zeolite framework structure.

FIG. 4B shows an SEM-image of the crystalline product obtained according to Example 3.

Thus, as demonstrated for Example 2, the additional acidification step performed in Example 4 compared to Example 3 leads to a product which may be easily filtered and washed, making the step of acidification particularly interesting for industrial scale production and the handling of large amounts of product.

### Example 5

15 g of an aqueous diethyldimethylammonium hydroxide solution (40 wt.-%) and 0.2 g of sodium hydroxide were mixed together and stirred for 20 min, after which 0.78 g of aluminum hydroxide were added and the mixture stirred a further 20 min, after which 6 g of fumed silica (Cab-O-Sil M5) were then added. The mixture was then transferred to a 150 ml Teflon lined autoclave in which it was crystallized under autogenous pressure at 150° C. for 3 d, during which the reaction vessel was tumbled at 20 rpm. After having let the reaction mixture cool to room temperature, the reaction product was centrifuged at 4 krpm for 50 min, and the white solid repeatedly elutriated with distilled water. The washed solid was then isolated by centrifugation and dried at 100° C. for 16 h, after which the resulting white powder was calcined at 600° C. for 10 h.

The surface area of the crystalline product of Example 5 was investigated by nitrogen absorption (cf. DIN 66135), wherein the evaluation of the data afforded a BET surface area of 678 m²/g.

The resulting product was analyzed by inductively coupled plasma mass spectrometry (ICP-MS), thus affording in Si:Al ratio of 11.

### Example 6

The procedure of Example 5 was repeated, wherein the mixture was crystallized for 7 d.

The surface area of the crystalline product of Example 6 was investigated by nitrogen absorption (cf. DIN 66135), wherein the evaluation of the data afforded a BET surface area of 669 m²/g.

The crystalline product of Example 6 was analyzed by inductively coupled plasma mass spectrometry (ICP-MS), thus affording in Si:Al ratio of 9.0.

### Example 7

The procedure of Example 5 was repeated, wherein the mixture was crystallized for 13 d.

The surface area of the crystalline product of Example 7 was investigated by nitrogen absorption (cf. DIN 66135), wherein the evaluation of the data afforded a BET surface area of 787 m²/g.

The crystalline product of Example 7 was analyzed by inductively coupled plasma mass spectrometry (ICP-MS), thus affording in Si:Al ratio of 8.6.

1. A process for the production of a zeolitic material having an LEV-type framework structure comprising YO₂ and optionally comprising X₂O₃, wherein said process comprises:
(1) preparing a mixture comprising one or more sources for YO₂, one or more solvents, and optionally comprising seed crystals; and
(2) crystallizing the mixture obtained in step (1);
wherein Y is a tetravalent element, and X is a trivalent element,
wherein the zeolitic material optionally comprises one or more alkali metals M, wherein the molar ratio of the total amount of the one or more solvents to the total amount of the one or more sources for YO₂ based on YO₂ is 9.5 or less, and
wherein for crystallization temperatures of 175° C. or higher in step (2), the duration of crystallization at those temperatures is less than 14 d.

2. The process of claim 1, wherein the one or more solvents comprise one or more polar solvents.

3. The process of claim 1, wherein the mixture in step (1) further comprises one or more organotemplates.

4. The process of claim 1, wherein the molar ratio of the total amount of the one or more organotemplates to YO₂ of the mixture obtained in step (1) ranges from 0.01 to 2.

5. The process of claim 1, wherein Y is selected from the group consisting of Si, Sn, Ti, Zr, Ge, and mixtures of two or more thereof.

6. The process of claim 1, wherein X is selected from the group consisting of Al, B, In, Ga, and mixtures of two or more thereof.

7. The process of claim 1, wherein the one or more sources for YO₂ comprises silica.

8. The process of claim 1, wherein the mixture in step (1) further comprises one or more sources for X₂O₃.

9. The process of claim 8, wherein the one or more sources for X₂O₃ comprises one or more aluminum compounds.

10. The process of claim 8, wherein the YO₂:X₂O₃ molar ratio of the mixture obtained in step (1) ranges from 2 to 200.

11. The process of claim 1, wherein the mixture according to step (1) further comprises one or more sources for OH⁻.

12. The process of claim 11, wherein the OH⁻:YO₂ molar ratio of the mixture obtained in step (1) ranges from 0.01 to 5.

13. The process of claim 1, wherein the mixture according to step (1) further comprises one or more sources of one or more elements suitable for isomorphous substitution of at least a portion of the Y atoms and/or of the X atoms in the LEV-type framework structure.

14. The process of claim 13, wherein the molar ratio of YO₂ to the total amount of the one or more elements suitable for isomorphous substitution of at least a portion of the Y atoms and/or of the X atoms in the LEV-type framework structure ranges from 3 to 300.

15. The process of claim 1, wherein the zeolitic material comprises one or more alkali metals M, and wherein the M:YO₂ molar ratio of the mixture obtained in step (1) ranges from 0.005 to 1.

16. The process of claim 1, wherein the mixture in step (1) further comprises one or more sources for X₂O₃, wherein the zeolitic material comprises one or more alkali metals M, and wherein the YO₂:X₂O₃:M molar ratios of the mixture obtained in step (1) range from 1: (0.005-1): (0.005-1).

17. The process of claim 1, wherein the crystallization in step (2) involves heating of the mixture.

18. The process of claim 17, wherein the crystallization in step (2) is conducted under solvothermal conditions.

19. The process of claim 17, wherein the crystallization in step (2) involves heating of the mixture for at least 0.1 d.

20. The process of claim 1, wherein the crystallization in step (2) involves agitating the mixture.

21. The process of claim 1, further comprising one or more of the following:
(3) isolating the zeolitic material having an LEV-type framework structure, preferably by filtration, ultrafiltration, diafiltration, centrifugation and/or decantation methods,
and/or
(4) washing the zeolitic material having an LEV-type framework structure,
and/or
(5) drying and/or calcining the zeolitic material having an LEV-type framework structure,
and/or
(6) subjecting the zeolitic material having an LEV-type framework structure to an ion-exchange procedure,
wherein the steps (3) and/or (4) and/or (5) and/or (6) can be conducted in any order.

22. The process of claim 21, wherein the calcination in step (5) is conducted at a temperature in the range of from 300 to 900° C.

23. The process of claim 21, wherein after step (2) and prior to step (3) the pH of the crystallization product is adjusted to a pH in the range of from 5 to 12.

24. The process of claim 21, wherein in the one or more step (6) at least one ionic non-framework element contained in the zeolitic material having an LEV-type framework is ion-exchanged.

25. The process of claim 1, wherein the zeolitic material having an LEV-type framework structure formed in step (2) comprises one or more zeolites selected from the group consisting of Levyne, LZ-132, NU-3, RUB-1, ZK-20, ZSM-45, RUB-50, and mixtures of two or more thereof.

26. The process of claim 1, wherein the seed crystals at least partially comprise zeolitic material having an LEV-type framework structure.

27. The process of claim 26, wherein the zeolitic material having an LEV-type framework structure contained in the seed crystals comprises one or more zeolites selected from the group consisting of Levyne, LZ-132, NU-3, RUB-1, ZK-20, ZSM-45, RUB-50, and mixtures of two or more thereof.

28. The process of claim 1, wherein the seed crystals at least partially comprise zeolitic material not having an LEV-type framework structure.

29. The process of claim 28, wherein the zeolitic material having a CHA-type framework structure contained in the seed crystals comprises chabazite and/or SSZ-13.

30. The process of claim 1, wherein the amount of seed crystals in the mixture according to step (1) ranges from 0.01 to 30 wt.-% based on 100 wt.-% of YO₂ in the at least one source for YO₂.

31. The process of claim 1, wherein the mixture according to step (1) contains 5 wt.-% or less of seed crystals based on 100 wt.-% of YO₂.

32. A zeolitic material having an LEV-type framework structure obtainable according to the process of claim 1.

33. A zeolitic material having an LEV-type framework structure, said zeolitic material comprising YO₂ and X₂O₃,
wherein Y is a tetravalent element, and X is a trivalent element, wherein the zeolitic material optionally comprises one or more alkali metals M, and
wherein the zeolitic material displays an Y:X atomic ratio of from 1 to 9.4.

34. The zeolitic material of claim 33, wherein Y is selected from the group consisting of Si, Sn, Ti, Zr, Ge, and mixtures of two or more thereof.

35. The zeolitic material of claim 33, wherein X is selected from the group consisting of Al, B, In, Ga, and mixtures of two or more thereof.

36. The zeolitic material of claim 33, said material having an X-ray diffraction pattern comprising at least the following reflections:

| Intensity (%) | Diffraction angle 2θ/° [Cu K(alpha 1)] |
|---|---|
| [26-40] | [10.94-11.20] |
| [23-27] | [13.36-13.62] |
| [50-66] | [17.33-17.62] |
| [42-57] | [21.05-21.37] |
| 100 | [22.08-22.21] |
| [35-56] | [28.59-28.92] |
| [48-62] | [32.37-32.60] |
| [12-19] | [51.60-52.14] |
| [11-13] | [55.68-56.32] |

wherein 100% relates to the intensity of the maximum peak in the X-ray powder diffraction pattern.

37. The zeolitic material of claim 33, wherein at least a portion of the Y atoms and/or of the X atoms in the LEV-type framework structure is isomorphously substituted by one or more elements.

38. The zeolitic material of claim 37, wherein the molar ratio of YO₂ to the total amount of the one or more elements by which the LEV-type framework structure is isomorphously substituted ranges from 5 to 100.

39. The zeolitic material of any of claim 33, wherein the zeolitic material comprises one or more alkali metals M, and wherein at least a portion of the alkali metal atoms M is substituted by one or more cation and/or cationic element.

40. The zeolitic material of claim 33, wherein said material comprises one or more zeolites selected from the group consisting of Levyne, LZ-132, NU-3, RUB-1, ZK-20, ZSM-45, RUB-50, and mixtures of two or more thereof.

41. A method of catalyzing a chemical reaction comprising the step of contacting one or more chemical compounds with the zeolitic material according to claim 33.
